(19) United States
(12) Patent Application Publication
(10) Pub. No.: US 2026/0215793 A1
(43) Pub. Date: Jul. 30, 2026

(54) CATHETER HAVING RADIOPAQUE MARKERS IN A FUNNEL MOUTH DISTAL END OF THE CATHETER

(71) Applicant: Neuravi Limited, Galway (IE)

(72) Inventors: Karl KEATING, Galway (IE); Declan LEE, Longford (IE); Artur SPYCHALA, Galway (IE); Avril O'HIGGINS, Galway (IE); Sinead KENNY, Galway (IE); David VALE, Barna (IE)

(73) Assignee: Neuravi Limited, Galway (IE)

(21) Appl. No.: 19/423,161

(22) Filed: Dec. 17, 2025

Related U.S. Application Data
(63) Continuation-in-part of application No. 18/912,912, filed on Oct. 11, 2024.
(60) Provisional application No. 63/599,805, filed on Nov. 16, 2023.

(51) Int. Cl. A61B 17/221; A61B 90/00; A61B 17/22

(52) U.S. Cl. CPC A61B 17/221; A61B 90/39; A61B 2017/22051; A61B 2090/3966

(57) ABSTRACT

The designs and methods disclosed herein are for a clot retrieval catheter with a large bore shaft and a distal braid-supported tip that is expandable to a diameter larger than the outer sheath through which it is delivered. The shaft can have a plurality of supporting braids fixed distally to a radiopaque marker band. The tip can have another plurality of supporting braids fixed proximally to the marker band and a decreasing braid angle distally so that the tip can be heat set to an expanded funnel shape. The wires of the tip braids can follow one spiral direction distally and then invert proximally back on themselves to form the other spiral direction of the braid. This inversion of the wires results in atraumatic distal hoops at the distal termination of the braid. Designs can further have spines capable of resisting elongation of the catheter shaft during a procedure.

### CROSS REFERENCE TO RELATED APPLICATIONS

The present disclosure is a continuation-in-part of U.S. Pat. App. No. 18/912,912 filed October 11, 2024 (Attorney Docket No.: 243382.000594 (NRV6145USNP1)), which application claims the benefit of priority under 35 U.S.C. § 119 to prior filed U.S. Provisional Patent Application No. 63/599,805, filed November 16, 2023 (Attorney Docket No.: 243382.000513 (NRV6145USPSP1)), the entire contents of which are hereby incorporated by reference as if set forth in full herein.

### FIELD OF INVENTION

The present disclosure generally relates devices and methods for removing acute blockages from blood vessels during intravascular medical treatments. More specifically, the present disclosure relates to retrieval catheters with expandable tips having radiopaque markers into which an object or objects can be retrieved.

### BACKGROUND

Clot retrieval aspiration catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed in particular is challenging with conventional technology, as the target vessels are small in diameter, remote relative to the site of insertion, and highly tortuous. These catheters are frequently of great length and must follow the configuration of the blood vessels in respect of all branching and windings. Traditional devices are often either too large in profile, lack the deliverability and flexibility needed to navigate particularly tortuous vessels, or are not effective at removing a clot when delivered to the target site.

Many existing designs for aspiration retrieval catheters are often restricted to, for example, inner diameters of 6Fr or between approximately 0.068 – 0.074 inches. Larger sizes require a larger guide or sheath to be used, which then necessitates a larger femoral access hole to close. Most physicians would prefer to use an 8Fr guide / 6Fr sheath combination, and few would be comfortable going beyond a 9Fr guide / 7Fr sheath combination. This means that once at the target site, a clot can often be larger in size than the inner diameter of the aspiration catheter and must otherwise be immediately compressed to enter the catheter mouth. This compression can lead to bunching up and subsequent shearing of the clot during retrieval. Firm, fibrin-rich clots can also become lodged in the fixed-mouth tip of these catheters making them more difficult to extract. This lodging can also result in shearing where softer portions breaking away from firmer regions of the clot.

Small diameters and fixed tip sizes are also less efficient at directing the aspiration necessary to remove blood and thrombus material during the procedure. The suction must be strong enough such that any fragmentation that may occur as a result of aspiration or the use of a mechanical thrombectomy device cannot migrate and occlude distal vessels. When aspirating with a fixed-mouth catheter, a significant portion of the aspiration flow ends up coming from vessel fluid proximal to the tip of the catheter, where there is no clot. This significantly reduces aspiration efficiency, lowering the success rate of clot removal.

Large bore intermediate and aspiration catheters and/or those with expandable tips are therefore desirable because they provide a large lumen and distal mouth to accept a clot with minimal resistance. The bore lumen of these catheters can be nearly as large as the guide and/or sheath through which they are delivered, and the expandable tip can expand to be a larger diameter still. When a clot is captured and drawn proximally into a tip with a funnel shape, the clot can be progressively compressed during retrieval so that it can be aspirated fully through the catheter and into a syringe or cannister. Funnel shaped catheters also lend themselves to better alignment in a vessel, as when the size of the tip is close to that of a vessel the device can self-center. The smaller diameter just proximal of the funnel portion of the tip can allow a hinging motion for bending between the tip and the shaft, in contrast to fixed-mouth designs where there is less freedom of motion and the tip and shaft will tend to move together.

In many examples, the fixed-mouth catheters and those with expandable tips can have an underlying braid as the primary supporting backbone. The use of braids in a catheter body is not a novel concept, and typical examples can be readily found in the art. The braid can often be as simple as bands wrapped spirally in one direction for the length of the catheter which cross over and under bands spiraled in the opposite direction. The bands can be metallic, fiberglass, or other material providing effective hoop strength to reinforce the softer outer materials of the body. However, supporting braids can often lack an effective bonding mechanism for the layers, or have a high sectional stiffness the point where they do not meet the flexibility criteria for many procedures. Additionally, many of these devices have structures which cannot be made soft enough for use in fragile vessels without causing substantial trauma. Furthermore, these devices often lack radiopaque markers in the distal expandable tip to aid the user when tracking or navigating particularly tortuous vessels. If the device has markers in the distal expandable tip, the markers effect the softness and flexibility of the catheter, especially at its distal or leading end when navigating within the vessels.

Combining the clinical needs of these catheters without significant tradeoffs can be tricky. Catheter designs attempting to overcome the above-mentioned design challenges would need to have a large bore and an expandable tip with sufficient hoop strength in the expanded state to resist aspiration forces without collapse while having a structure capable of folding down consistently and repeatably when retrieved into an outer guide and/or sheath. The tip structure needs to have the flexibility and elasticity to survive the severe mechanical strains imparted when navigating the tortuous vasculature, while also being capable of expanding elastically as a clot is ingested for better interaction with and retention of the clot. The tip structure also needs to have radiopaque markers so the user will know where the catheter, including its distal end, are while navigating in tortuous vessels.

Axially, the tip shape with radiopaque markers must maintain good pushability so that it can be advanced within an outer sheath, and in the expanded state in a vessel with minimal tendency to further over-expand outward when placed in compression. The tip in the expanded state can also be advanced through vessels that are smaller in diameter, as the funnel shape allows the tip to radially compress when advanced through a narrowing vessel with minimal force. This is advantageous as the tip can seal in a wider range of vessel sizes.

As a result, there remains a need for improved catheter designs attempting to overcome the above-mentioned design challenges. The presently disclosed designs are aimed at providing an improved retrieval catheter with an expansile tip and methods for fabricating such a catheter capable improved performance.

### SUMMARY

It is an object of the present designs to provide devices and methods to meet the above-stated needs. The designs can be for a clot retrieval catheter capable of remove a clot from cerebral arteries in patients suffering AIS, from coronary native or graft vessels in patients suffering from MI, and from pulmonary arteries in patients suffering from PE and from other peripheral arterial and venous vessels in which a clot is causing an occlusion. The designs can also resolve the challenges of aspirating fibrin rich clot material by addressing the key difficulties of 1) the friction between the clot and the catheter and 2) the energy/work required to deform these firm clots as they are aspirated into the catheter tip.

In some examples, a catheter can include a liner having a proximal end, a distal end, an outer surface, an internal lumen extending from the proximal end to the distal end, and a longitudinal axis. A braid is formed by at least one wire disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape that extends from the proximal end to proximate the distal end. The braid has a tapered shape portion at the distal end of the braid. The tapered shape portion of the braid gradually increases in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells being disposed at the distal end of the braid proximal to and adjacent to the second circumferential ring of cells. A fourth circumferential ring of cells is disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each being in the tapered shape portion of the braid. At least one radiopaque marker is disposed within one of the second, third, fourth, fifth, and sixth circumferential rings of cells. A jacket is disposed about the braid. The jacket has a proximal end and a distal end. The jacket extends from the proximal end of the liner to the distal end of the liner. The distal end of the jacket is aligned with distal end of the liner. The distal end of the braid is spaced proximally from the distal end of the liner and from the distal end of the jacket.

In some other examples, the catheter can include a liner having a proximal end, a distal end, an outer surface, an internal lumen extending from the proximal end to the distal end, and a longitudinal axis. A braid is disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape portion extending from the proximal end to proximate the distal end. The braid has a tapered shape portion at the distal end of the braid. The tapered shape portion of the braid gradually increasing in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells is disposed at the distal end of the braid proximal to and adjacent to the second circumferential ring of cells. A fourth circumferential ring of cells being disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each are disposed in the tapered shape portion of the braid. The distal end of the braid has distal loops where at least one wire of the braid inverts and forms the circumferential rings of cells. At least one radiopaque marker is disposed within one of the distal loops. A jacket is disposed about the braid. The jacket has a proximal end and a distal end. The jacket extends from the proximal end of the liner to the distal end of the liner. The distal end of the jacket is aligned with distal end of the liner.

In some other examples, the catheter can include a liner having a proximal end, a distal end, an outer surface, an internal lumen extending from the proximal end to the distal end, and a longitudinal axis. A braid is disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape extending from the proximal end to proximate the distal end. The braid has a tapered shape portion at the distal end of the braid. The tapered shape portion of the braid gradually increasing in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells is disposed at the distal end of the braid proximal to and adjacent to the second circumferential ring of cells. A fourth circumferential ring of cells is disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each are in the tapered shape portion of the braid. The braid has a plurality of wires that create the circumferential rings of cells. The distal end of the braid has free ends. At least one radiopaque marker is disposed about at least one of the free ends of the braid. A jacket is disposed about the braid. The jacket has a proximal end and a distal end. The jacket extends from the proximal end of the liner to the distal end of the liner. The distal end of the jacket is aligned with distal end of the liner.

In some other examples, the catheter can include a liner that has a proximal end, a distal end, an outer surface, an internal lumen extending from the proximal end to the distal end, and a longitudinal axis. A braid is made of a wire disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape portion extending from the proximal end to proximate the distal end. The braid has a tapered shape portion at the distal end of the braid. The tapered shape portion of the braid gradually increasing in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells is disposed at the distal end of the braid proximal to and adjacent to the second circumferential ring of cells. A fourth circumferential ring of cells is disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each are in the tapered shape portion of the braid. At least one radiopaque marker is disposed about at least one wire of the braid within one of the first, second, third, fourth, fifth, and sixth circumferential rings of cells. A jacket is disposed about the braid. The jacket has a proximal end and a distal end. The jacket extends from the proximal end of the liner to the distal end of the liner. The distal end of the jacket is aligned with distal end of the liner. The distal end of the braid is spaced proximally from the distal end of the liner and from the distal end of the jacket.

In some other examples, the catheter can include a liner having a proximal end, a distal end, an outer surface, an internal lumen extending from the proximal end to the distal end, and a longitudinal axis. A braid is made of wire disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape extending from the proximal end to proximate the distal end. The braid has a tapered shape at the distal end of the braid. The tapered shape of the braid gradually increasing in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells is disposed at the distal end of the braid proximal to and adjacent to the second circumferential ring of cells. A fourth circumferential ring of cells is disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each are disposed in the tapered shape portion of the braid. A jacket is disposed about the braid. The jacket has a proximal end and a distal end. The jacket extends from the proximal end of the liner to the distal end of the liner. The distal end of the jacket is aligned with distal end of the liner. The distal end of the braid is spaced proximally from the distal end of the liner and from the distal end of the jacket. A tip is disposed at the distal end of the catheter. The tip is connected to the jacket. The tip is made of a polymer material that is impregnated with a radiopaque material.

In some other examples, the catheter can include a braid that is disposed on the outer surface of the liner. The braid has a proximal end and a distal end. The braid extends from the proximal end of the liner to the distal end of the liner. The distal end of the braid is spaced from and proximal to the distal end of the liner. The braid has a tubular shape extending from the proximal end to proximate the distal end. The braid has a tapered shape at the distal end of the braid. The tapered shape of the braid gradually increases in diameter in the distal direction. The braid forms a plurality of circumferential rings of cells. A first circumferential ring of cells is disposed at the distal end of the braid. A second circumferential ring of cells is disposed at the distal end of the braid proximal to and adjacent to the first circumferential ring of cells. A third circumferential ring of cells is disposed at the distal end of the braid proximal to and adjacent to the second circumferential ring of cells. A fourth circumferential ring of cells is disposed at the distal end of the braid proximal to and adjacent to the third circumferential ring of cells. A fifth circumferential ring of cells is disposed at the distal end of the braid proximal to and adjacent to the fourth circumferential ring of cells. A sixth circumferential ring of cells is disposed at the distal end of the braid proximal to and adjacent to the fifth circumferential ring of cells. The first, second, third, fourth, fifth, and sixth circumferential ring of cells each are disposed in the tapered shape portion of the braid. A jacket is disposed about the braid. The jacket has a proximal end and a distal end. The jacket extends from the proximal end of the liner to the distal end of the liner. The distal end of the jacket is aligned with distal end of the liner. The distal end of the braid is spaced proximally from the distal end of the liner and from the distal end of the jacket. At least one radiopaque marker is disposed between the braid and the jacket within one of the second, third, fourth, fifth, and sixth circumferential rings of cells. The at least one radiopaque marker is disposed in at least two cells of the braid and under a wire of the braid that separates the at least two cells.

In some other examples, a method for manufacturing a catheter is disclosed. The method can include providing a liner having a proximal end, a distal end, an outer surface, an internal lumen extending from the proximal end to the distal end, and a longitudinal axis. The outer surface of the liner can be etched. A braid can be provided on the outer surface of the liner. The braid has a proximal end and a distal end. The braid forms a plurality of diamond shaped cells. The braid extends from the proximal end of the liner to the distal end of the liner. The distal end of the braid is spaced from and proximal to the distal end of the liner. A first sleeve is placed about the distal end of the braid covering at least a distalmost circumferential row of diamond shaped cells. The first sleeve has a proximal end and a distal end. The first sleeve prevents UV light from passing through the sleeve. At least one radiopaque marker is placed on the outer surface of the liner within at least one of the diamond shaped cells adjacent to and spaced a predetermined distance from the distal end of the braid and spaced proximally from the proximal end of the first sleeve. The at least one radiopaque marker is connected with an adhesive to the at least one radiopaque marker 140. The at least one radiopaque marker is placed within a circumferential ring of cells proximal of the proximal end of the first sleeve. The first sleeve is connected to the distal end of the braid. The first sleeve is pulled in the distal direction, so the braid is flush with the liner. An ultraviolet (UV) light source is provided. The adhesive is cured by directing UV light from the UV light source toward the at least one radiopaque marker after the pulling the first sleeve step. At least one jacket is placed about the braid. The jacket has a proximal end and a distal end. The first sleeve is removed from the distal end of the braid after the curing step. The jacket is reflowed about the braid, the at least one radiopaque marker, and the liner to form the catheter.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combine elements from multiple figures to better suit the needs of the user.

FIG. 1 is a plan view of a large bore clot retrieval catheter attached to a connection hub with an expandable tip being disposed within a sheath configured for advancing through the vasculature according to aspects of the present disclosure;

FIG. 2 illustrates the distal portion of a large bore clot retrieval catheter with an expandable tip according to aspects of the present disclosure;

FIG. 2A is a view of a large bore clot retrieval catheter with an expandable tip being advanced through the vasculature, according to aspects of the present disclosure;

FIG. 3 shows a cutaway view, with parts broken away, of a large bore clot retrieval catheter according to aspects of the present disclosure;

FIG. 4 shows a cutaway view of a large bore clot retrieval catheter according to aspects of the present disclosure;

FIG. 4A is a cross-sectional view taken along lines 4A-4A in FIG. 4 and looking in the direction of the arrows;

FIG. 5 shows a cutaway view of a large bore clot retrieval catheter according to aspects of the present disclosure;

FIG. 6 illustrates an example braid configuration for the distal tip section according to aspects of the present disclosure;

FIG. 7 is another example of a braid configuration for the distal tip section according to aspects of the present disclosure;

FIG. 8 is a view of the distal tip section of FIG. 7 according to aspects of the present disclosure;

FIG. 9 is another example of a braid configuration for the distal tip section according to aspects of the present disclosure;

FIG. 10 is a view of the distal tip section of FIG. 9 according to aspects of the present disclosure;

FIG. 11 is another example of a braid configuration for the distal tip section according to aspects of the present disclosure;

FIG. 12 is another example of a braid configuration for the distal tip section according to aspects of the present disclosure;

FIGS. 13A-D are perspective views of a marker for use with the distal tip section of the catheter illustrated in FIG. 12;

FIG. 14 is another example of a braid configuration for the distal tip section according to aspects of the present disclosure;

FIG. 15A is a perspective view of a marker for use with the distal tip section of the catheter illustrated in FIG. 14;

FIG. 15B is an end view of the marker of FIG. 15A for use with the distal tip section of the catheter illustrated in FIG. 14;

FIG. 16 is another example of a braid configuration for the distal tip section according to aspects of the present disclosure;

FIG. 17A is a perspective view of a marker for use with the distal tip section of the catheter illustrated in FIG. 16;

FIG. 17B is an end view of the marker of FIG. 15A for use with the distal tip section of the catheter illustrated in FIG. 16;

FIG. 18 is another example of a braid configuration for the distal tip section according to aspects of the present disclosure;

FIG. 19A is a perspective view of a marker for use with the distal tip section of the catheter illustrated in FIG. 18;

FIG. 19B is an end view of the marker of FIG. 15A for use with the distal tip section of the catheter illustrated in FIG. 18;

FIG. 20A is another example of a braid configuration for the distal tip section according to aspects of the present disclosure;

FIG. 20B is a partial enlarged cross-sectional view taken along lines 20B-20B of FIG. 20A and looking in the direction of the arrows.

FIG. 21 is a partial enlarged cross-sectional view taken along lines 21-21 of FIG. 20A and looking in the direction of the arrows.

FIG. 22 is an enlarged view of the distal tip section with a marker being shown of the catheter illustrated in FIG. 20A;

FIG. 23 is an enlarged view of the distal tip section with a marker being shown of the catheter illustrated in FIG. 20A;

FIG. 24 is a plan view of a marker for use with the distal tip section of the catheter illustrated in FIG. 20A;

FIG. 25 is a partial plan view of a first sleeve being disposed about the distal end of a braid and covering at least a distalmost circumferential row of diamond shaped cells of a catheter according to aspects of the present disclosure;

FIG. 25A is a partial enlarged cross-sectional view taken along lines 25A-25A of FIG. 25 and looking in the direction of the arrows.

FIG. 25B is a cross-sectional view taken along lines 25B-25B of FIG. 25 and looking in the direction of the arrows.

FIG. 26 is a partial plan view of a first sleeve being disposed about the distal end of a braid and covering at least a distalmost circumferential row of diamond shaped cells of a catheter according to aspects of the present disclosure;

FIG. 27 is a partial plan view of a first sleeve being disposed about the distal end of a braid and covering at least a distalmost circumferential row of diamond shaped cells of a catheter according to aspects of the present disclosure;

FIGS. 28A and 28B are another example of a braid configuration for the distal tip section according to aspects of the present disclosure;

FIG. 29 is a plan view of a catheter and an ultraviolet (UV) light source for curing an adhesive to connect a marker to a liner according to aspects of the present disclosure;

FIG. 30 is a plan view of a catheter disposed on a mandrel for reflowing the jacket according to aspects of the present disclosure;

FIG. 31 is a plan view of a catheter disposed on a mandrel for reflowing the jacket according to aspects of the present disclosure;

FIG. 32 is a plan view of a catheter disposed on a mandrel for reflowing the jacket according to aspects of the present disclosure;

FIG. 33 is a plan view of a catheter disposed on a mandrel for reflowing the jacket according to aspects of the present disclosure;

FIG. 34 is a plan view of a catheter disposed on a tapered mandrel within the distal end of the reflowed liner, braid, and jacket according to aspects of the present disclosure; and

FIG. 35 is a plan view of a catheter disposed on a tapered mandrel within the distal end of the reflowed liner, braid, and jacket according to aspects of the present disclosure.

### DETAILED DESCRIPTION

Specific examples of the present disclosure are now described in detail with reference to the Figures, where identical reference numbers indicate elements which are functionally similar or identical. The examples address many of the deficiencies associated with traditional clot retrieval aspiration catheters, such as poor or inaccurate deployment to a target site and ineffective clot removal.

The designs herein can be for a super-bore clot retrieval catheter with a large internal lumen and a distal funnel tip that can self-expand to a diameter larger than that of the guide or sheath through which it is coaxially delivered. The designs can have a proximal elongate body for the shaft of the catheter, and a distal tip with an expanding braided support structure and outer polymeric jacket to give the tip atraumatic properties. The braided support can be designed so that the expansion capability is variably focused in an axial portion of the tip section. The braid cells can be capable of easily and repeatably collapsing for delivery and expanding for good clot reception and resistance under aspiration. Sections of the tip can have the ability to further expand beyond the free shape of the expanded deployed configuration when ingesting a clot. The catheter’s braid and tip designs can be sufficiently flexible to navigate highly tortuous areas of the anatomy and be able to recover its shape to maintain the inner diameter of the lumen when displaced in a vessel. The design of the tip structure also has radiopaque markers so the user will know where the catheter, including its distal end, are while navigating in the tortuous vessels. The markers are designed to have minimal effect on the softness and flexibility of the catheter, especially at its distal or leading end when navigating within the vessels.

Accessing the various vessels within the vascular, whether they are coronary, pulmonary, or cerebral, involves well-known procedural steps and the use of a number of conventional, commercially-available accessory products. These products, such as angiographic materials, mechanical thrombectomy devices, microcatheters, and guidewires are widely used in laboratory and medical procedures. When these products are employed in conjunction with the devices and methods of this disclosure in the description below, their function and exact constitution are not described in detail. Additionally, while the description is in many cases in the context of thrombectomy treatments in intercranial arteries, the disclosure may be adapted for other procedures and in other body passageways as well.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

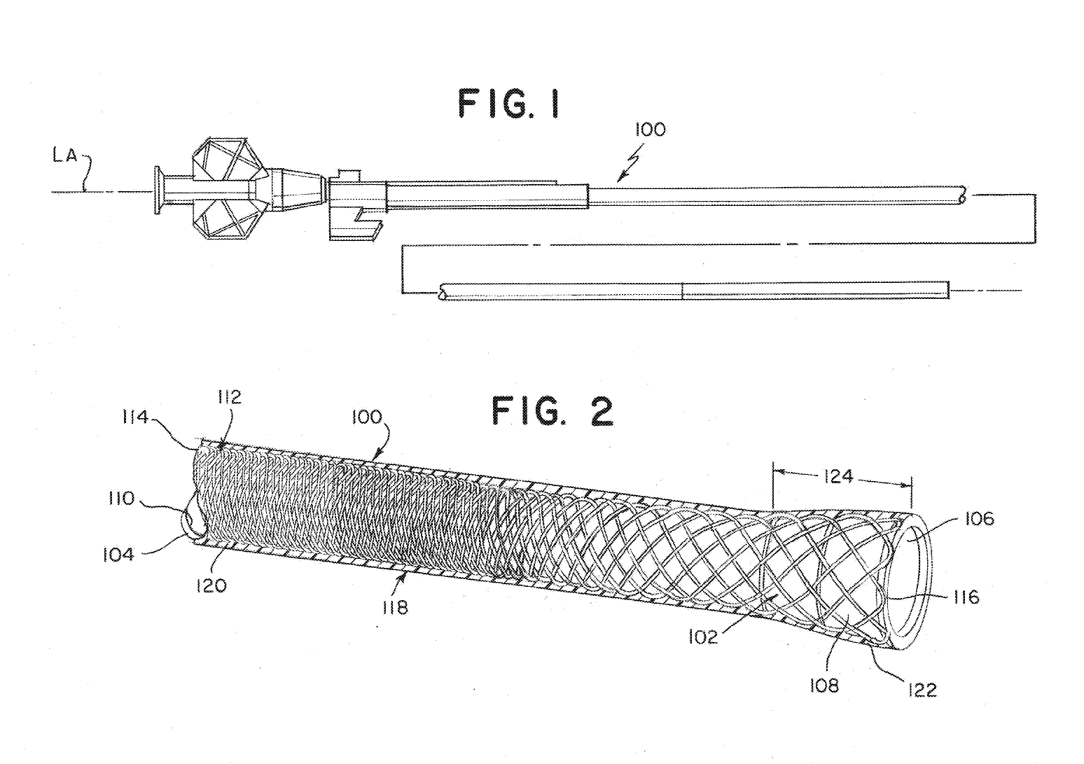

Turning to the figures, FIG. 1 illustrates a catheter 100 attached to a connection hub 400, according to aspects of the present disclosure. The catheter 100 can be a system including a catheter connection hub 400 connected to the proximal end 112 of the elongate shaft 110. The connection hub 400 has a strain relief extension 402 extending therefrom. The connection hub 400 has a proximal locking mechanism 404 configured to engage with a known aspiration device. The strain relief extension 402 is a length of polymeric tubing that extends from within the connection hub 400 to a position distal to the hub 400 and over a length of the proximate elongate shaft 110 to prevent the proximate elongate shaft 110 from crimping or breaking near the connection hub 400. The strain relief extension 402 can have a hardness of approximately 45–65 Shore D, for example 55 Shore D. The strain relief extension 402 can extend distally over the elongate shaft 110 for a length of approximately 30mm, or for example anywhere from 15–20 mm, 20–25 mm, 25–30 mm, 30–35 mm, 35–40 mm, etc. The proximal locking mechanism 404 can be a threaded connection or any other type of connection to connect the catheter 100 to an auxiliary device.

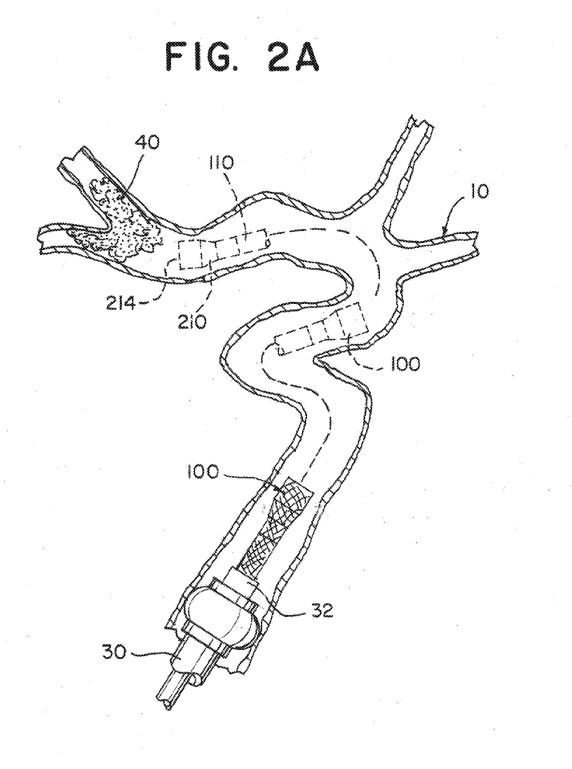

FIG. 2A illustrates a large bore clot retrieval catheter 100 of the designs disclosed herein. The clot 40 can be approached with the catheter 100 collapsed within a guide sheath or other outer catheter for delivery. When the vasculature 10 becomes too narrow and/or tortuous for further distal navigation with the guide sheath 30, the catheter 100 can be deployed for further independent travel distally. The catheter 100 can be highly flexible such that it is capable of navigating the M1 or other tortuous regions of the neurovascular to reach an occlusive clot.

Referring now to FIG. 2, the clot retrieval catheter 100 can have a flexible elongate body 110 serving as a shaft with a large internal bore and a distal tip section 210 having a collapsible supporting braided structure. The large bore helps the catheter to be delivered to a target site by a variety of methods. These can include over a guidewire, over a microcatheter, with a dilator/access tool, or by itself. The inner diameter (ID) of the elongate body 110 can be, for example, approximately 0.071 inches, and the ID of catheter 100 at the distal end of the tip section 210 can be, for example, approximately 0.092 inches. In some examples of the present disclosure, the ID of the elongate body 110 and of the distal end of the tip section 210 can be larger or smaller. For example, the ID of the elongate body 110 can be, for example, approximately 0.092 inches, and the ID of catheter 100 at the distal end of the tip section 210 can be, for example, approximately 0.125 inches. Alternatively, the ID of the elongate body 110 can be, for example, approximately 0.057 inches, and the ID of catheter 100 at the distal end of the tip section 210 can be, for example, approximately 0.078 inches. In another alternative, the ID of the elongate body 110 can be, for example, approximately 0.042 inches, and the ID of catheter 100 at the distal end of the tip section 210 can be, for example, approximately 0.062 inches. Thus, the ID of the elongate body 110 can range from approximately 0.092 inches to 0.042 inches, and the ID of catheter 100 at the distal end of the tip section 210 can range from approximately 0.125 inches to approximately 0.062 inches. In most cases, the design of the collapsible funnel tip can be configured so that the catheter 100 can be delivered through (and retrieved back through) commonly sized outer sheaths and guides. For example, a standard 6Fr sheath / 8Fr guide, would typically have an inner lumen of less than 0.090 inches. The tip can then be designed with a collapsed delivery outer diameter of approximately 0.086 inches. Of course, if the ID of the guide sheath is 0.090”, the outer diameter (OD) of the tip will collapse to about 0.090” and not to its maximum collapsed OD of approximately 0.086 inches. The tip can self-expand once advanced to an unconstrained position distal to the distal end 32 of the guide sheath 30, capable of reaching expanded an OD of approximately 0.132 inches. In other examples of the disclosure, the expanded OD can be approximately 0.140 inches or 0.150 inches. As the catheter can be delivered independently to a remote occlusion, the tip section 210 must be designed to be able to resist collapse from the forces of aspiration, have excellent lateral flexibility in both the expanded and collapsed states, an atraumatic profile to prevent snagging on bifurcations in vessels, and conformability to allow self-sizing should the tip need to be advanced through vessels with a diameter smaller than the tip and track past calcified plaque without dislodging it.

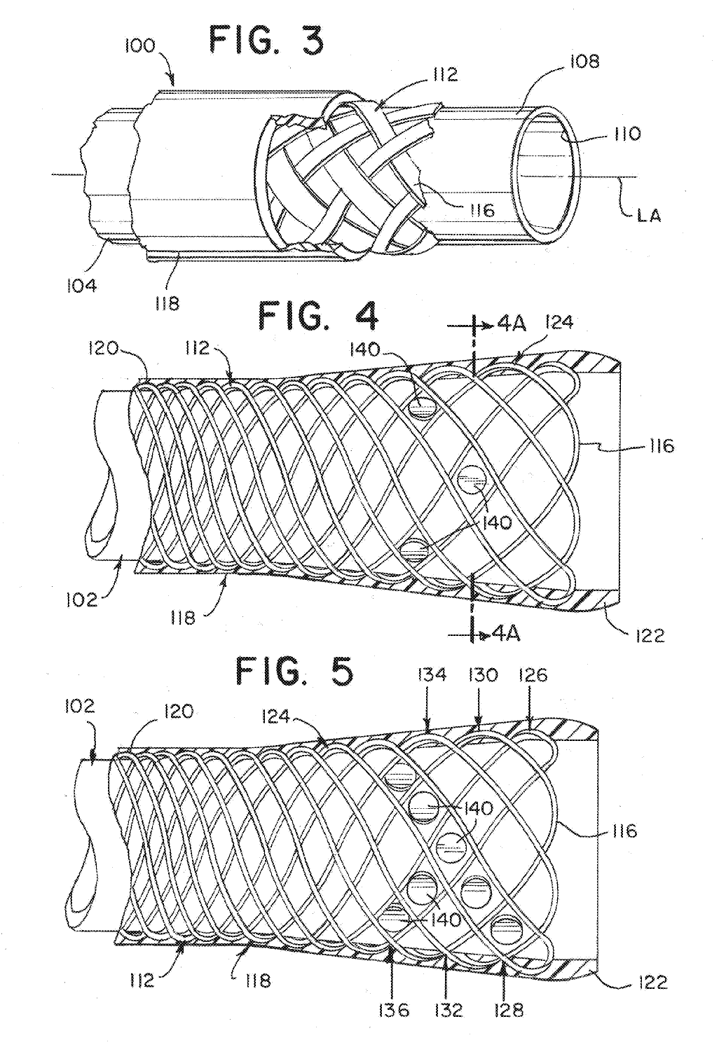

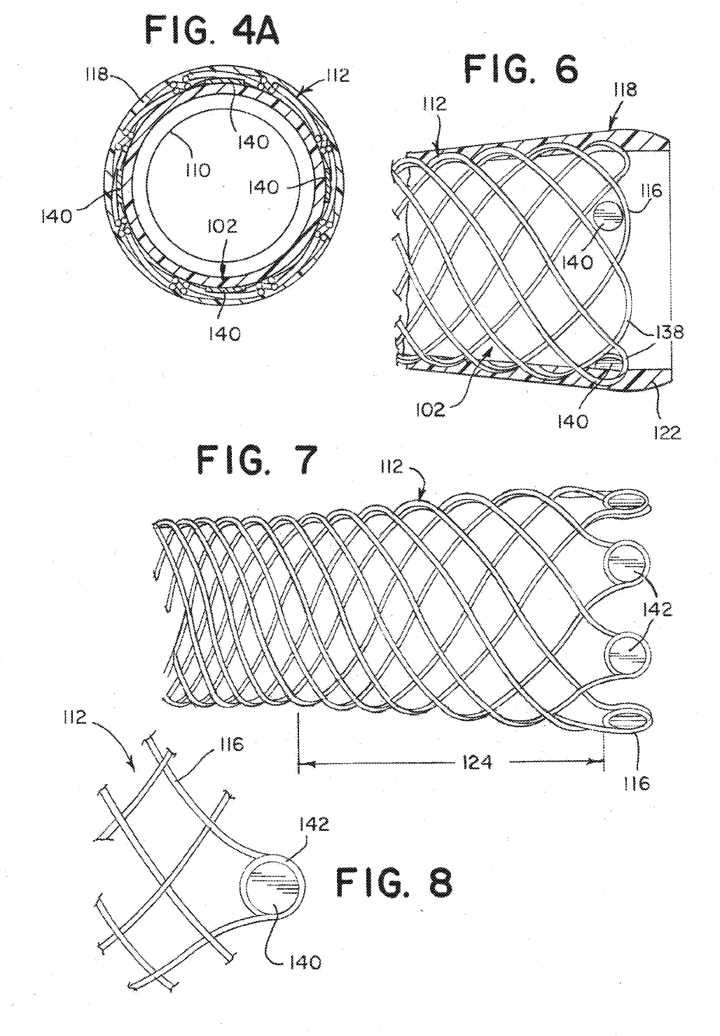

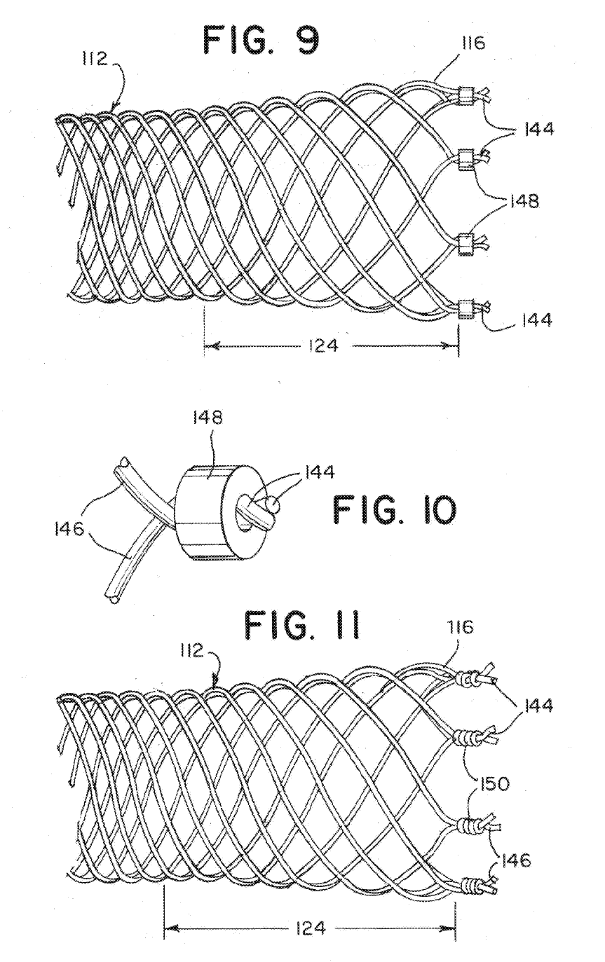

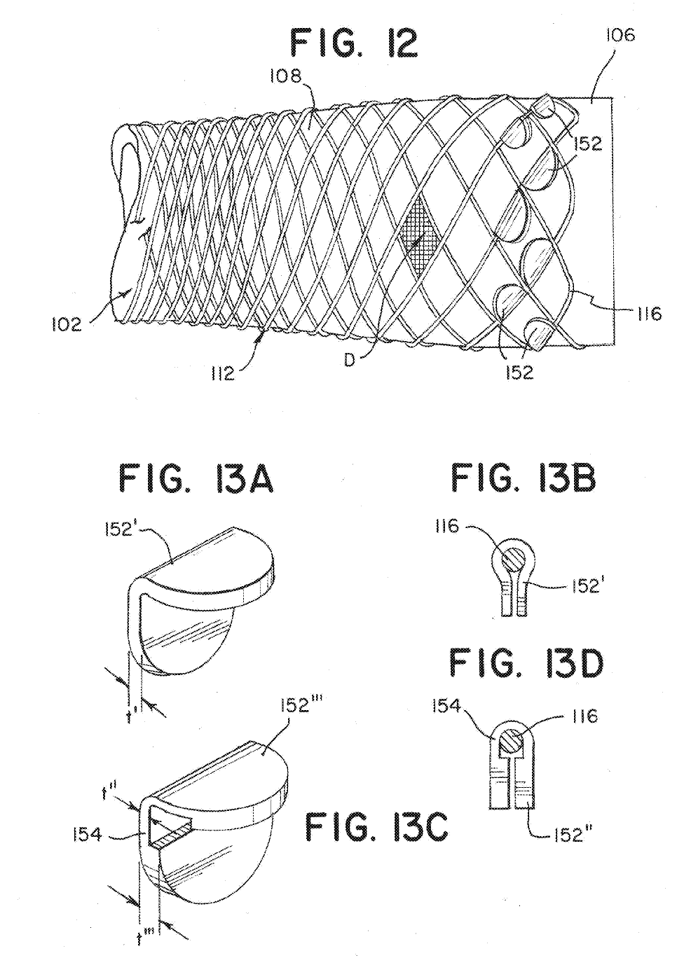

Referring now to FIGS. 3-6, a cutaway view, with parts broken away, of a large bore clot retrieval catheter 100 according to aspects of the present disclosure is shown in FIG. 3. Catheter 100 includes a liner 102 that has a proximal end 104, a distal end 106, and an outer surface 108. Catheter 100 has an internal lumen 110 extending from the proximal end 104 to the distal end 106 along a longitudinal axis LA. A braid 112 is formed by at least one wire disposed on the outer surface 108 of the liner 102. The braid 112 has a proximal end 114 and a distal end 116. Braid 112 extends from the proximal end 104 of the liner 102 to the distal end 106 of the liner 102. The distal end of the braid 116 is preferably spaced from and proximal to the distal end 106 of the liner 102, as shown for example, in FIGS. 3-6. In accordance with one example of the disclosure, the distal end 116 of the braid 112 is spaced proximally from the distal end 106 of the liner 102 and from the distal end of the jacket 118 by approximately 0.50 mm. Braid 112 has a tubular shape that extends from the proximal end 114 to proximate the distal end. Braid 112 has a tapered shape portion 124 at the distal end 116 of the braid 112. The tapered shape portion 124 of the braid gradually increases in diameter in the distal direction. Referring now to FIG. 5, braid 112 forms a plurality of circumferential rings of cells 126, 128, 130, 132, 134, and 136. A first circumferential ring of cells 126 is disposed at the distal end of the braid. A second circumferential ring of cells 128 is disposed at the distal end of the braid proximal to and adjacent to the first circumferential ring of cells 126. A third 130 circumferential ring of cells is disposed at the distal end of the braid proximal to and adjacent to the second circumferential ring of cells 128. A fourth circumferential ring of cells 132 is disposed at the distal end of the braid proximal to and adjacent to the third circumferential ring of cells 130. A fifth circumferential ring of cells 134 is disposed at the distal end of the braid proximal to and adjacent to the fourth circumferential ring of cells 132. A sixth circumferential ring of cells 136 is disposed at the distal end of the braid proximal to and adjacent to the fifth circumferential ring of cells 134. The first, second, third, fourth, fifth, and sixth circumferential ring of cells each are disposed in the tapered shape portion 124 of the braid 112. The tapered shape portion 124 of the braid is comprised of approximately between 6 and 16 rings of cells. In one example, there can be about thirteen circumferential rings of cells.

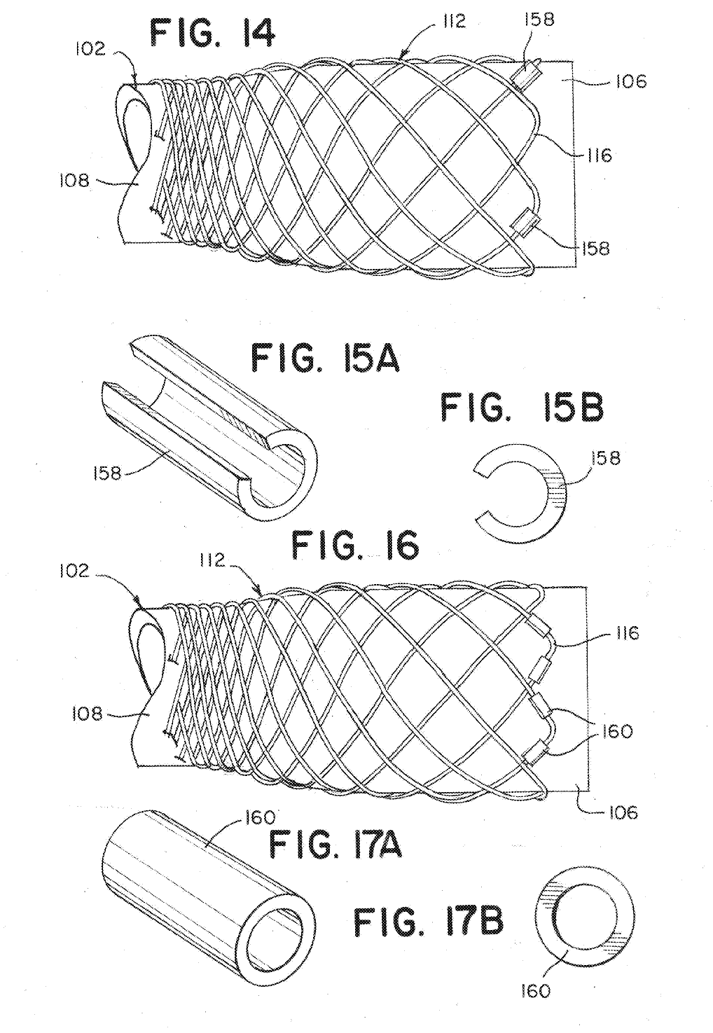

As shown in FIGS. 4-6, at least one radiopaque marker 140 is disposed within one of the second 128, third 130, fourth 132, fifth 134, and sixth 136 circumferential rings of cells. The radiopaque markers 140 are each preferably disc shaped. At least one radiopaque marker 140 disposed within one of the third 130, fourth 132, fifth 134, and sixth 136 circumferential rings of cells. At least two radiopaque markers 140 are positioned within the fourth 132 circumferential ring of cells defined by the distalmost cells. As shown in FIG. 4, four radiopaque markers 140 are positioned within the fourth 132 circumferential ring of cells spaced at approximately 90 degrees apart within the fourth circumferential rings of cells. Additionally, four radiopaque markers 140 are positioned within the sixth 136 circumferential ring of cells spaced at approximately 90 degrees apart within the fourth circumferential rings of cells, as shown in FIG. 4. As shown in FIG. 6, the distal end 116 of the braid 112 has a plurality of distal hoops or bends 138 where the at least one wire of the braid inverts to loop back through the braid and form the circumferential rings of cells. The distal hoops 138 form an atraumatic end at the distal end of braid 112.K

Referring now to FIGS. 7 and 8, another example of a braid configuration for the distal tip section according to aspects of the present disclosure is illustrated. When describing other examples, like numerals indicate like structural elements and features in various figures. Like elements and features in additional examples will not be described again for the sake of brevity in the disclosure. In accordance with the example illustrated in FIGS. 7 and 8, a jacket 118 is disposed about the braid 112. The distal end of the braid 112 is spaced proximally from the distal end 106 of the liner 102 and from the distal end of the jacket 118. The distal end 116 of the braid 112 comprises distal loops 142. In these loops 142, the wire of the braid inverts over 360º and forms the circumferential rings of cells 126, 128, 130, 132, 134, and 136. Loops 142 are circular in shape in accordance with this example but could have other shapes, such as oval, polygonal, etc. A disc-shaped radiopaque marker 140 can be disposed within one or more of the distal loops 142. In a current example, each loop 142 can receive a marker 140.

Referring now to FIGS. 9-11, another example of a braid configuration for the distal tip section according to aspects of the present disclosure is illustrated. In this example, the braid comprises a plurality of wires that create the circumferential rings of cells. The distal end 116 of the braid 112 comprising free ends 144. Radiopaque markers 148 are placed about the free ends of the twisted wires 146. In one example, the radiopaque marker 148 can be a sleeve that is crimped about the distal end of the braid as shown in FIG. 10. In another example, the marker 150 is formed from knotting the braid wire at the distal end of the braid as shown in FIG. 11. In other examples of the disclosure, a radiopaque element can be knotted about the distal end of the braid. However, in another example of the disclosure, if the braid wire is made of a radiopaque material, the radiopacity of the distal end of the catheter can be accentuated by tying the knot from the radiopaque braid wire to locally increase the wire density and hence the radiopacity.

Referring now to FIGS. 12-13D, another example of a braid configuration for the distal tip section according to aspects of the present disclosure is illustrated. In this example, at least one radiopaque marker 152 is disposed about at least one wire of the braid 112 within one of the first, second, third, fourth, fifth, and sixth circumferential rings of cells. In many examples, there are a plurality of markers 152 disposed about a wire of the braid. For example, as illustrated in FIG. 12, there can be two markers 152 placed about a wire for each diamond D in the second circumferential rings of cells. An example of a diamond D formed by the braided wire is shown in the highlighted diamond in FIG. 12. The radiopaque marker 152ʹ can have a planar shape that is then bent at an approximate 90º angle as illustrated in FIGS. 13A and 13C. A thickness tʹ of the radiopaque marker 152ʹ is about half the diameter of the wire forming the braid. The radiopaque marker 152ʹ is folded about the wire 116 of the braid 112, as illustrated in FIG. 13B. Because the thickness tʹ of the radiopaque marker 152ʹ is about half the diameter of the wire forming the braid, when the marker is folded about the wire there is a minimal increase in the profile of the braid. Marker 152ʹ can be crimped, glued or otherwise attached to wire 116. Once jacket 118 is reflowed over the braid, markers and liner, the markers will be firmly held in place embedded within the catheter 100. In addition, the overall OD of the catheter will not be increased due to the folded over marker because the reflowed jacket will accommodate the space taken by the marker over the braid wire. Referring now to FIGS. 13C and 13D, a thickness tʺ of a middle portion 154 of the radiopaque marker 152ʺ adjacent the wire of the braid is about 25% of the diameter of the wire forming the braid. A thickness tʺʹ of a portion of the radiopaque marker 152ʺadjacent the middle portion 156 of the radiopaque marker 152ʺ is about 75% of the diameter of the wire forming the braid. Like marker 152ʹ, marker 152ʺis folded about the wire 116 of the braid 112, as illustrated in FIG. 13D. Marker 152ʺ can be crimped, glued or otherwise attached to wire 116.

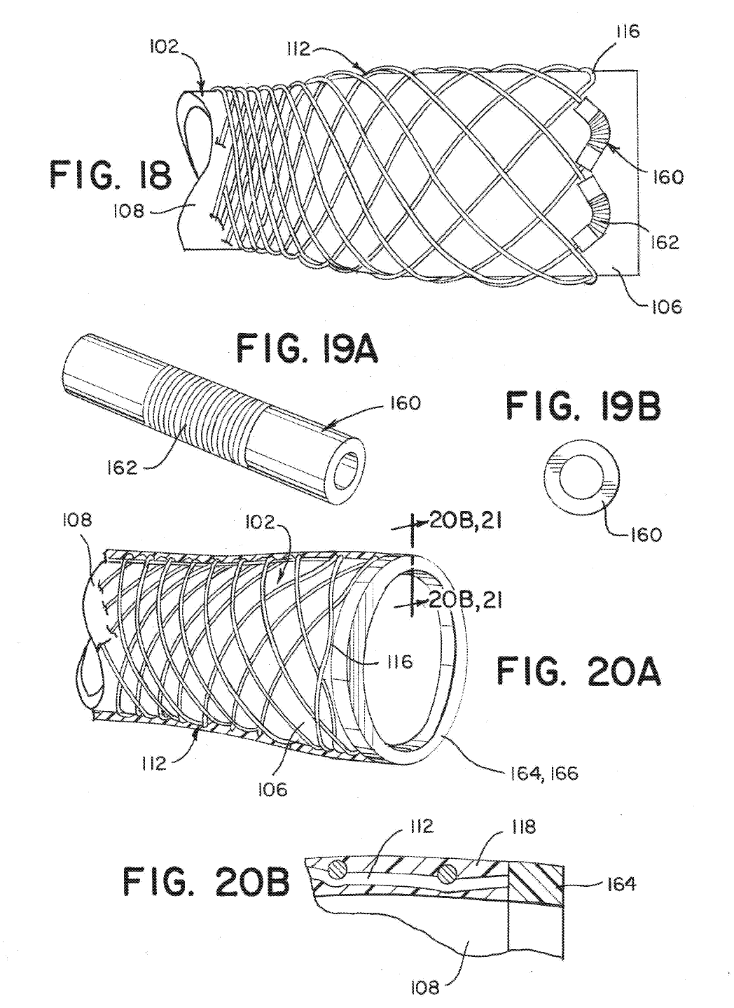

Referring now to FIGS. 14-19B, other examples of a braid configuration for the distal tip section according to aspects of the present disclosure is illustrated. Radiopaque marker 158 has a C-shape. Radiopaque marker 160 has a tubular shape. Radiopaque markers 158, 160 can be crimped onto the wire 116. Radiopaque marker 160 can have a plurality of circumferential grooves 162 in the center of the tubular shape configured to promote bending of the marker to follow the contour of the wire 116, as illustrated in FIGS. 18 and 19A.

Referring now to FIGS. 20A-21, other examples of a braid configuration for the distal tip section according to aspects of the present disclosure is illustrated. In this example, a tip 164, 166 is disposed at the distal end of the catheter. The tip is comprised of an inner layer 166 and an outer layer 164. The tip is connected to the jacket 118. The tip 164, 166 can be made from a polymer material that is impregnated with a radiopaque material. The radiopaque material can be, for example, either Tantalum and/or Tungsten. The tip can also be made from a thermoplastic polyurethane. Inner layer 166 can be impregnated with a radiopaque material. In one example, the inner layer 166 is impregnated with about 60% to about 90% Tantalum or Tungsten filler.

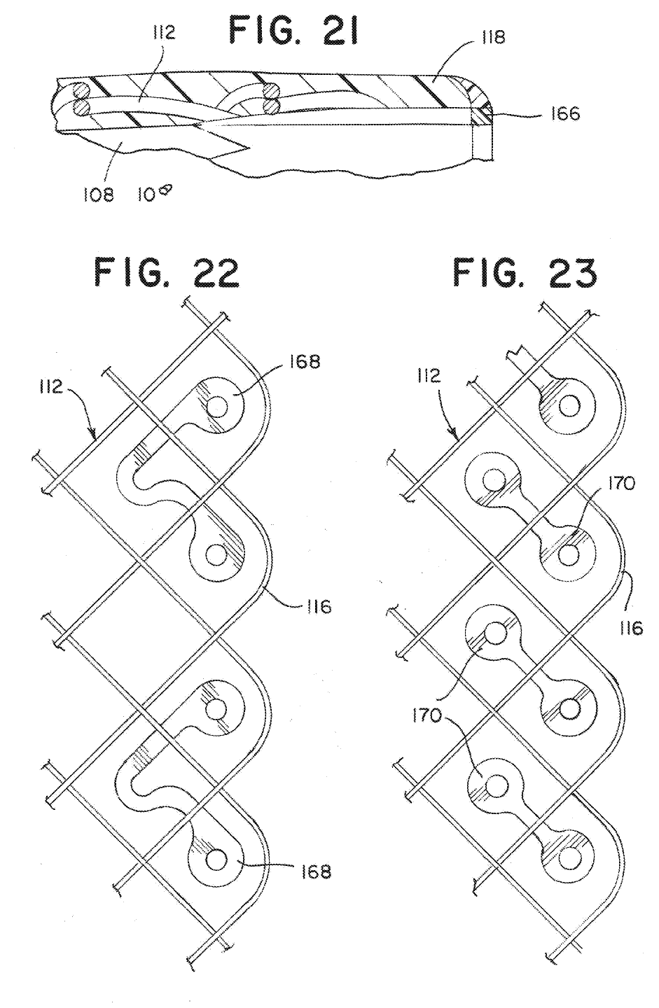

Referring now to FIGS. 22-24, other examples of a braid configuration for the distal tip section according to aspects of the present disclosure is illustrated. In this example, the radiopaque marker(s) 168, 170, 172 are disposed in at least two diamond shaped cells D of the braid and span under a wire 116 of the braid 112 that separates the at least two cells. As illustrated in FIGS. 23 and 24, marker 170, 172 can have a symmetrical shape. Alternatively, as illustrated in FIG. 22, marker 168 can have an asymmetrical shape. The marker 170, 172 can span under one wire and be disposed in two diamond shaped cells as shown in FIG. 23, or the marker 168 can span under two wires and be disposed in three diamond shaped cells as shown in FIG. 23.

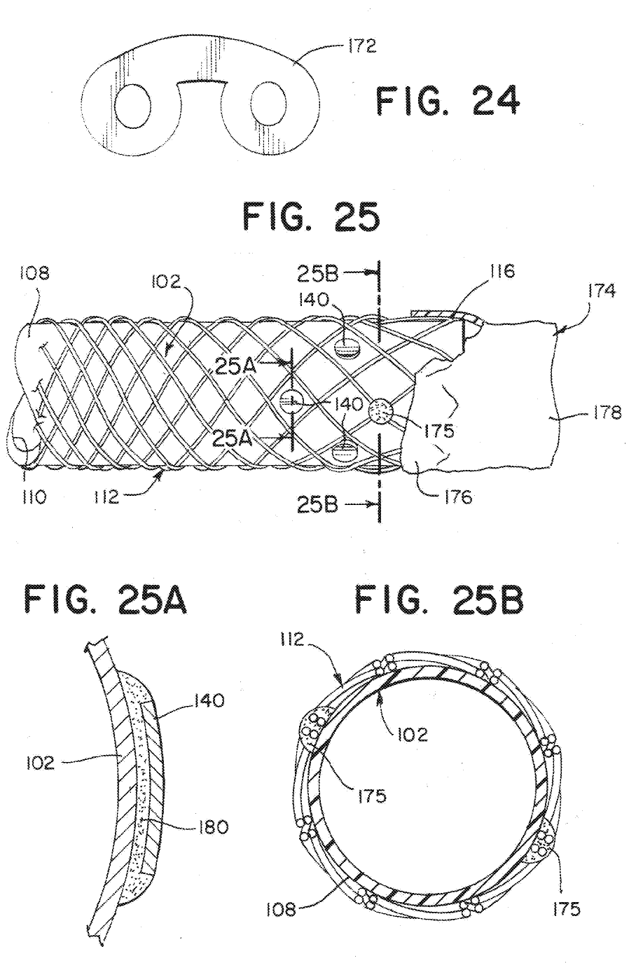

Referring now to FIGS. 25-33, an example of a method of making a catheter 100 for navigating within the vasculature of a body according to aspects of the present disclosure is illustrated. A liner 102 is provided in step 300. Liner 102 has a proximal end 104, a distal end 106, an outer surface 108, an internal lumen 110 extending from the proximal end 104 to the distal end 106, and a longitudinal axis LA.

In step 302, the outer surface 108 of the liner 102 is etched. In step 304, a braid 112 is provided on the outer surface 108 of the liner 102. Braid 112 has a proximal end 114 and a distal end 116. Braid 112 forms a plurality of diamond shaped cells D. Braid 112 extends from the proximal end 104 of the liner 102 to the distal end 106 of the liner 102. The distal end of the braid 116 is spaced from and proximal to the distal end 106 of the liner 102. In one example, the braid 112 can be formed on a stainless-steel mandrel, heat set, and then transferred onto the liner 102, which is supported on a copper core. Thereafter, the copper core can be stretched after the outer jacket(s) are reflowed about the braid and liner to allow removal of the now formed catheter 100.

In step 306, a first sleeve 174 is placed about the distal end of the braid. First sleeve 174 covers at least a distalmost circumferential row of diamond shaped cells. First sleeve 174 has a proximal end 176 and a distal end 178. First sleeve 174 prevents UV light from passing through the sleeve 174. Alternatively, if a UV adhesive is not used, then the PET sleeve does not need to block UV, but it can still be used to hold the braid down while gluing the markers, as discussed below. It is noted that in one example, before step 306, the proximal end of the braid is secured to the liner 102. For example, the proximal end of the braid can be glued to the liner and a jacket about the proximal end of the braid can be tacked in place by partially, but not fully, reflowing this jacket about the proximal end of the braid. In this example, the proximal portion of the braid is made from stainless-steel and this portion of the braid is connected or joined to a distal portion of the braid that is made of drawn-filled- tube (“DFT”) braid with a smooth transition between the two materials. The DFT portion of the braid is made of a super-elastic material, such as, for example nitinol or alloys thereof. If the braid is made from a single material, then the braid could secure using a thin PET sleeve or by twisting the braid wires over the proximal end of the copper core to hold the braid down until the reflowed jackets are able to hold the braid in place.

In step 308, at least one radiopaque marker 140 is placed on the outer surface of the liner 102 within at least one of the diamond shaped cells D adjacent to and spaced a predetermined distance from the distal end of the braid 112 and spaced proximally from the proximal end 176 of the first sleeve 174, as shown in FIG. 25.

In step 310, an adhesive 180 is used to connect the at least one radiopaque marker 140 to the liner 102, as shown in FIG. 25A. The at least one radiopaque marker 140 is placed within a circumferential ring of cells proximal of the proximal end 176 of the first sleeve 174. In addition, an adhesive may be used at the crossing 175 of the wires of the braid 112 at the 1.5 pick between circumferential rings as shown in FIGS. 25 and 25B, to hold the braid down. In one example, the adhesive is applied at two wire crossings 180 degrees apart from one another about the braid, as shown in FIG. 25B. Of course, more or less wire crossings can be glued down to the liner. For example, in one example, the adhesive is applied at four wire crossings 90 degrees apart from one another about the braid.

In step 312, first sleeve 174 is connected to the distal end 116 of the braid 112.

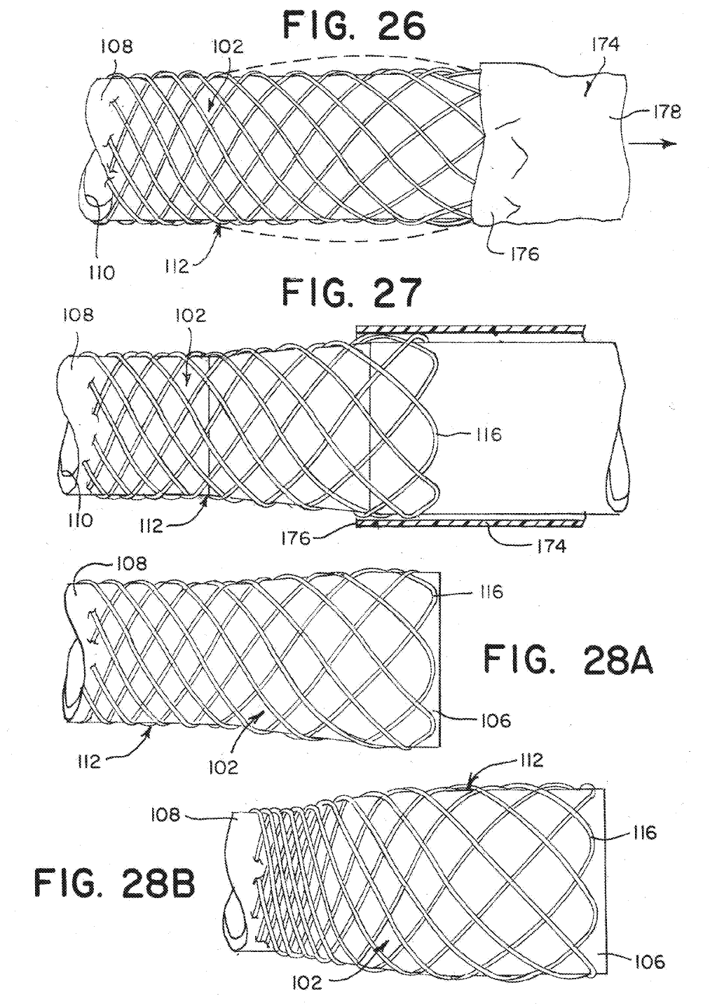

In step 314, first sleeve 174 is pulled in the distal direction so the braid 112 is flush with the liner 102, as illustrated in FIG. 26. In one example of this disclosure, the sleeve may first be stretched to pull the braid down onto the mandrel, and then the markers 140 can be placed within a circumferential ring of cells proximal of the proximal end 176 of the first sleeve 174. The markers 140 can then be glued in place. In this manner, braid 112 will not move over the markers 140 if stretching is down after positioning the markers 140

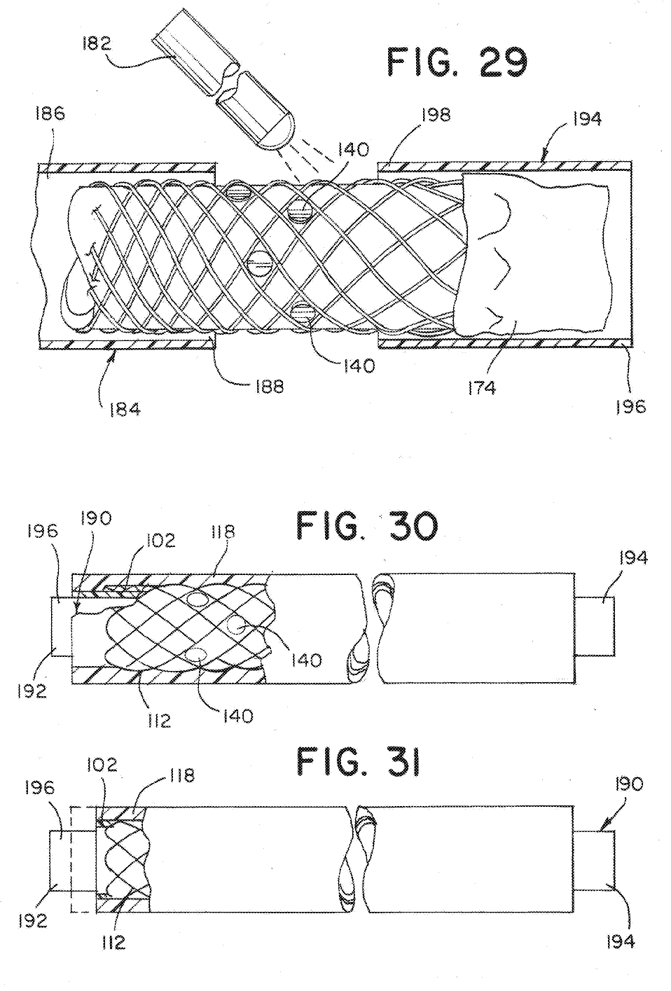

In step 316, an ultraviolet (UV) light source 182 is provided. In step 318, the adhesive is cured by directing UV light from the UV light source 182 toward the at least one radiopaque marker 140 after the pulling the first sleeve step 314, as illustrated in FIG. 29.

In step 320, at least one jacket 118 is placed about the braid 112. Jacket 118 has a proximal end 120 and a distal end 122.

In step 322, first sleeve 174 is removed from the distal end of the braid 112 after the curing step.

In step 324, jacket 118 is reflowed about the braid 112, the at least one radiopaque marker 140, and the liner 102 to form the catheter 100. In another example of this disclosure, the jacket can also be tacked down over the braid and markers while making sure to not tack over the sleeve. In this manner, the braid is prevented from recoiling after the sleeve is removed. Once the sleeve is removed, the last two cells can be tacked down in place. All the proximal jackets can then be added and tacked in place before moving on to the final reflow step where you fully melt and reflow the polymer onto the PTFE liner. Another alternative to this process, includes using a very small amount of adhesive over the first pick or the second pick. Of course, all of the picks in the full circumferential rings of pick 2s could be tacked down in place, but in some examples, only two or three of the pick 2s need to be tacked down. The two or three pick 2s are preferably evenly distributed across the circumference of the braid.

In step 326, a second sleeve 184 is placed about the distal end of the braid 112 before the curing step. Second sleeve 184 is spaced proximal of the first sleeve 174 such that the at least one radiopaque marker 140 is disposed between the first sleeve 174 and the second sleeve 184. Second sleeve 184 has a proximal end 186 and a distal end 188. Distal end 188 of the second sleeve is proximal to and spaced from the most proximal of the at least one radiopaque markers 140 on the outer surface of the liner 102. Second sleeve 184 prevents UV light from passing through the sleeve, just like first sleeve 174. UV light is being blocked to prevent the UV light from degrading the etch of the PTFE liner which could affect lamination (adhesion) between the reflowed jacket and the outer diameter of the liner.

In step 328, second sleeve 184 is removed from the braid 112 after the curing step 318.

The reflowing of the jacket about the braid step 324 includes reflowing the jacket 118 from the distal end 122 of the jacket 118 up to a location proximal of the first sleeve 174.

The step of removing the first sleeve 322 from the distal end of the braid 112 occurs after the reflowing of the jacket 118 about the braid 112 from the proximal end 120 of the jacket 118 to a location proximal of the first sleeve 174.

In step 330, a remaining portion of the jacket is reflowed about the braid 112 from the location proximal of the first sleeve 174 to the distal end 122 of the jacket 118.

In step 332, a core 190 is provided. Core has a proximal end 192, a distal end 194, and an outer surface 196. The providing a liner step 304 includes placing the liner 102 on the outer surface of the core 190, as illustrated in FIG. 30.

In step 334, the liner 102 and jacket 118 are trimmed after the reflowing of a remaining portion of the jacket step 330. The liner 102 and jacket 118 are trimmed at a location distal to the distal end of the braid 112. In an example of the disclosure, the liner 102 and jacket are first trimmed on the copper core, but because the material is relatively soft, the liner and jacket can be trimmed on the copper core distal of the intended final distal cut so that the liner and jacket are too long. The liner and jacket can then be loaded onto a similar sized stainless-steel mandrel and trimmed to the final distal cut at a tighter tolerance.

In step 336, the reflowed liner 102, braid 112, and jacket 118 are removed from the core 190.

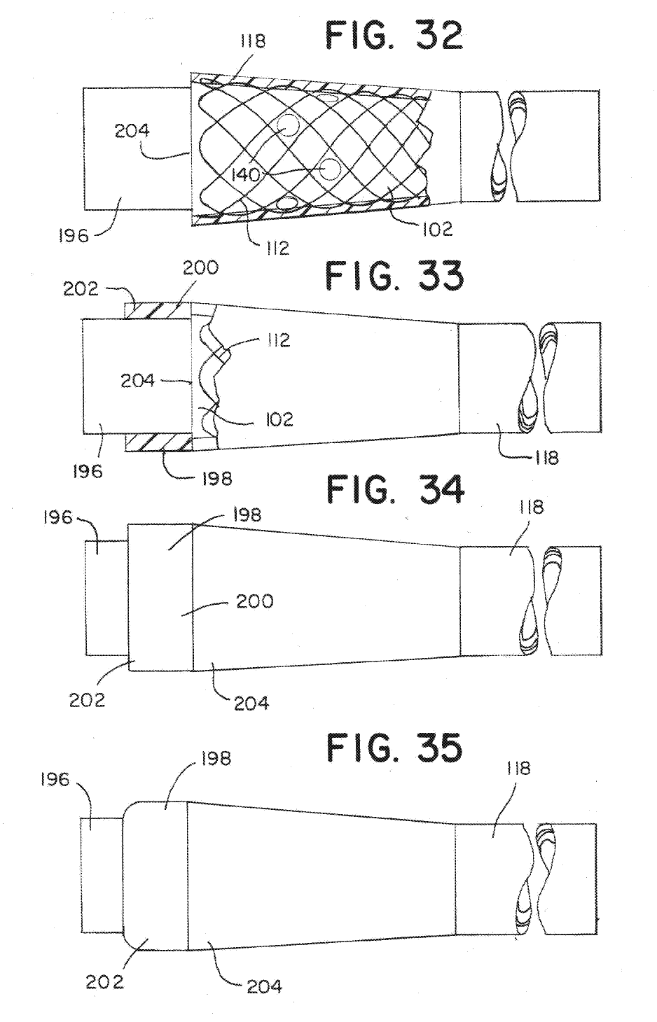

In step 338, a tapered mandrel 196 is placed within the distal end of the reflowed liner, braid, and jacket, as shown in FIG. 32. The distal tip is flared radially outwardly by pushing the reflowed liner, braid, and jacket a pre-determined distance over the tapered section of mandrel 196.

In step placing 340, a tubular sleeve 198 is placed about the tapered mandrel 196. Tubular sleeve 198 has a proximal end 200 and a distal end 202. The proximal end 200 of the tubular sleeve 198 abuts a distal end 204 of the reflowed liner and jacket, as illustrated in FIG. 33. In one example of the disclosure, tubular sleeve 198 can be preloaded onto the larger diameter end of tapered mandrel 196 before flaring the tip in previous step 338. In this manner, tubular sleeve 198 will not have to be pushed axially too far along the larger diameter end of tapered mandrel 196. Sleeve 198 can be sized to have a smaller inner diameter than the outer diameter of the larger diameter end of mandrel 196 so that sleeve 198 will grip onto the outer surface of mandrel 196. Of course, sleeve 198 could have a slightly larger inner diameter such that there is a small amount of clearance between the inner diameter of sleeve 198 and the outer diameter of mandrel 196, but this clearance would still need to be pretty tight to obtain a smooth transition to the flared section distal end 204 of the reflowed liner and jacket.

In step reflowing 342, the tubular sleeve 198 and at least a distal portion of the jacket 118 about the tapered mandrel 196 are reflowed to join sleeve 198 with jacket 118.

In step trimming 344, the tubular sleeve 198 is trimmed to form a rounded distal tip 199 at the distal end of the catheter 100, as illustrated in FIG. 35. In one example of the disclosure, the tubular sleeve 198 can be trimmed after reflow step 342 to cut the sleeve to the correct length (approximately 0.25mm to approximately 0.75mm). Before being cut, tubular sleeve 198 could be about 5mm or 10mm in axial length to make handling easier as it can be difficult to handle and moving around a short 0.25mm piece. Once tubular sleeve 198 is trimmed to the correct length, another reflow step can be used to form the rounded distal tip end 199. This final reflow step could be done using a tipping dye. In some examples, the final reflow step may cause the tip to extend a little too long. In such an example, the distal end of tubular sleeve 198 will need another trimming step to remove any excess material to form the rounded distal tip 199.

The placing a first sleeve 174 about the distal end of the braid step 306, includes covering at least two distalmost circumferential rows or rings of diamond shaped cells 126, 128. Sleeve 174 can be made of a material that inherently blocks UV radiation or can include a UV protective filler material. Sleeve 174 can be made from a heat shrink material. In one example of the disclosure, sleeve 174 can be made from a polyethylene terephthalate (PET) material. Sleeve 174 can also be made from polycarbonate, which has an inherent ability to block UV radiation. Other materials that sleeve 174 can be made from include: acrylic, which inherently has UV resistance; polyethylene terephthalate glycol (PETG), which has a moderate level of UV protection; polyethylene naphthalate (PEN), which absorbs UV light up to 390nm. Alternatively, sleeve 174 can include a UV additive that can be added to clear plastics such as PET, polypropylene (PP), polycarbonate and acrylic. A UV additive can be compounded with UV blockers like benzotriazoles or benzophenones to absorb UV. Alternatively, amber glass can be used as the amber color itself shields both UV and visible light. In an example of the disclosure, sleeve 174 can be made of PP or a high-density polyethylene compounded with an amber colored pigment or a UV blocking additive.

When using a catheter of the present disclosure to clear an occlusion from a body vessel, the super-bore catheter with an expandable/collapsible tip can be delivered through an outer catheter or sheath to a location proximal of a vessel occlusion. The outer catheter is typically placed as close to the occlusive clot as practical, but the location can depend on the destination vessel size and the relative tortuosity of the vasculature needed to reach it. For example, in the case of a middle cerebral artery occlusion, the outer catheter might be placed in the internal carotid artery proximal of the carotid siphon. If for example the target occlusion is in an M1 vessel, a typical guide or outer sheath will need to be maintained in a position well proximal of these vessel diameters. The outer catheter would likely fit in a proximal M1 vessel, however, the design of a guide catheter is typically too stiff to track tortuous vessels, but the stiffness is needed to give support around the aortic arch and to prevent the inner aspiration catheter from causing the guide catheter to back out (prolapse) of the common carotid artery.

For example, if an 8Fr collapsible super-bore catheter with a funnel inner diameter of 0.120 inches is used to aspirate and retrieve a large clot in the Internal Carotid Artery and branches (or a 6Fr catheter in the M1 vessels), the user then injects contrast to check vessel patency. If the user finds that some clot fragments remain in more distal vessels, the collapsible super-bore catheter can be advanced to aspirate the fragments in the more distant location. If access cannot be achieved due to tortuosity, the funnel outer diameter has reached a point where it matches the inner diameter of the vessels, or if a self-sizing funnel has reached the lower end of its vessel range, an inner catheter with a smaller diameter 6Fr (or 5fr) low shear tip can be rapidly advanced telescopically to the more distal vessel location to retrieve the clot through aspiration or co-aspiration with a stent retriever. As the inner catheter and clot is retracted through the super-bore catheter, the plunger-type suction effect of retracting one within the other can act to aspirate through the mouth of the super-bore catheter to ensure that any fragments that may have dislodged from the initial clot retrieval process are retrieved. Aspiration can also be directly applied to the lumen of the super-bore catheter so that a negative flow through it can be maintained during the procedure.

Consistent collapsibility of the expandable funnel tip is important for durability and ease of use. For example, when first loading into an outer sheath for delivery, the catheter can be advanced through an insertion tool that has no profile but has a longitudinal slit and a proximal flared section so you can funnel the catheter into the insertion tool. In another example, a profiled insertion tool can be used that will uniformly collapse the tip to fit inside an outer sheath such as a balloon guide catheter. The inner diameter of the collapsed tip is maintained so that smaller introducers or microcraters and guidewires can be advanced through it. This is advantageous when using a telescoping approach to reach a target vessel location. Telescoping allows the user to first advance a smaller more trackable catheter to the treatment location and then advance a larger catheter over it, using the smaller catheter/guidewire as a rail to guide advancement of the larger catheter. An alternative can be to advance a smaller fixed mouth catheter through the expandable tip catheter if vessel size prohibits its further advancement. The larger sized catheter can then act as a backup for retracting the smaller diameter catheter should a stiff clot become lodged in the shaft of the smaller catheter.

As another option, a loading tool can be supplied on the shaft of the catheter so that it is advanced distally over the funnel of the expandable tip to collapse the tip before inserting through the luer of an outer guide sheath. In another similar example, a split tool can be used to collapse the funnel through stretching. The tool halves can be joined together using thread locks, snap locks or with magnetic fastening features. This allows the user to attach, remove and reattach the tool as needed, for example, if the physician needs to do a second pass with the same catheter, the tool can be reattached to allow collapse of the flared tip for a second advancement through an outer guide after a first pass has been completed.

Once the target is reached, the occlusion can be aspirated and drawn into the tip and lumen of the expandable tip large bore catheter, or a smaller telescoping inner catheters where used, with the help of aspiration through one or multiple of the catheters in the system. If necessary, auxiliary devices such as microcatheters and clot retrieval devices can be advanced through the lumen of the large bore catheter and used against obstinate clots.

Once the clot has been dislodged from the vessel walls it can be progressively compressed through the funnel-shape of the expandable tip of the large bore catheter and through the lumen into a cannister or syringe. For a particularly firm clot, additional radial expansion of the tip section can protect and shelter a lodged clot while the catheter itself is withdrawn. Otherwise, the catheters can remain in position during this step to maintain access to the target site. Contrast can then be injected to determine the extent to which the vessel is patent. Clot retrieval devices may be rinsed in saline and gently cleaned before being reloaded into the guide catheter for additional passes, if required. When the vessel has been satisfactorily cleared, the catheter can be retracted to collapse the expandable tip within the outer catheter.

The disclosure is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near or a direction towards the physician. Furthermore, the singular forms “a,” “an,” and “the” include plural referents unless the context clearly dictates otherwise.

As used herein, the terms "about" or "approximately" for any numerical values or ranges indicate a suitable dimensional tolerance that allows the part or collection of components to function for its intended purpose as described herein. More specifically, "about" or "approximately" may refer to the range of values ±20% of the recited value, e.g. "about 90%" may refer to the range of values from 71% to 99%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the disclosure. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

Examples of the present disclosure can be implemented by any of the following numbered clauses:

Clause 1. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 formed by at least one wire disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid having a tubular shape extending from the proximal end 114 to proximate the distal end, the braid having a tapered shape portion 124 at the distal end 116 of the braid 112, the tapered shape portion 124 of the braid gradually increasing in diameter in the distal direction, the braid forming a plurality of circumferential rings of cells, a first circumferential ring of cells 126 being disposed at the distal end of the braid, a second circumferential ring of cells 128 being disposed at the distal end of the braid proximal to and adjacent to the first circumferential ring of cells, a third 130 circumferential ring of cells being disposed at the distal end of the braid proximal to and adjacent to the second circumferential ring of cells, a fourth circumferential ring of cells 132 being disposed at the distal end of the braid proximal to and adjacent to the third circumferential ring of cells, a fifth circumferential ring of cells 134 being disposed at the distal end of the braid proximal to and adjacent to the fourth circumferential ring of cells, a sixth circumferential ring of cells 136 being disposed at the distal end of the braid proximal to and adjacent to the fifth circumferential ring of cells, the first, second, third, fourth, fifth, and sixth circumferential ring of cells each being in the tapered shape portion 124 of the braid;

at least one radiopaque marker 140 disposed within one of the second 128, third 130, fourth 132, fifth 134, and sixth 136 circumferential rings of cells; and

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102; wherein the distal end of the braid is spaced proximally from the distal end 106 of the liner 102 and from the distal end of the jacket 118.

Clause 2. The catheter of Clause 1, wherein the at least one radiopaque marker 140 disposed within one of the third 130, fourth 132, fifth 134, and sixth 136 circumferential rings of cells.

Clause 3. The catheter of Clause 1, comprising at least two radiopaque markers 140 positioned within the fourth 132 circumferential ring of cells defined by the distalmost cells.

Clause 4. The catheter of Clause 3, wherein four radiopaque markers are positioned within the fourth 132 circumferential ring of cells at approximately 90 degrees apart within the fourth circumferential rings of cells.

Clause 5. The catheter of Clause 1 or 2, comprising at least two radiopaque markers positioned within the sixth 136 circumferential ring of cells defined by the distalmost cells.

Clause 6. The catheter of Clause 5, wherein four radiopaque markers are positioned within the sixth 136 circumferential ring of cells at approximately 90 degrees apart within the sixth circumferential rings of cells.

Clause 7. The catheter of Clause 1, wherein the distal end 116 of the braid 112 comprising a plurality of distal hoops 138 where the at least one wire of the braid inverts to loop back through the braid and form the circumferential rings of cells.

Clause 8. The catheter of any of Clauses 1-7, wherein the at least one radiopaque marker 140 is disc shaped.

Clause 9. The catheter of any of Clauses 1-8, wherein the tapered shape portion 124 of the braid is comprised of about thirteen circumferential rings of cells.

Clause 10. The catheter of any of Clauses 1-9, wherein the distal end 116 of the braid 112 is spaced proximally from the distal end 106 of the liner 102 and from the distal end of the jacket 118 by approximately 0.50 mm.

Clause 11. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid having a tubular shape portion extending from the proximal end 114 to proximate the distal end 116, the braid having a tapered shape portion 124 at the distal end 116 of the braid 112, the tapered shape portion 124 of the braid 112 gradually increasing in diameter in the distal direction, the braid forming a plurality of circumferential rings of cells, a first 126 circumferential ring of cells being disposed at the distal end of the braid, a second 128 circumferential ring of cells being disposed at the distal end of the braid proximal to and adjacent to the first circumferential ring of cells, a third 130 circumferential ring of cells being disposed at the distal end of the braid proximal to and adjacent to the second circumferential ring of cells, a fourth 132 circumferential ring of cells being disposed at the distal end of the braid proximal to and adjacent to the third circumferential ring of cells, a fifth 134 circumferential ring of cells being disposed at the distal end of the braid proximal to and adjacent to the fourth circumferential ring of cells, a sixth 136 circumferential ring of cells being disposed at the distal end of the braid proximal to and adjacent to the fifth circumferential ring of cells, the first, second, third, fourth, fifth, and sixth circumferential ring of cells each being in the tapered shape portion 124 of the braid, the distal end 116 of the braid 112 comprising distal loops 142 where at least one wire of the braid inverts and forms the circumferential rings of cells;

at least one radiopaque marker 140 disposed within one of the distal loops 142; and

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102.

Clause 12. The catheter of Clause 11, wherein the loops 142 are circular in shape.

Clause 13. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid having a tubular shape extending from the proximal end 114 to proximate the distal end 116, the braid having a tapered shape portion 124 at the distal end of the braid 112, the tapered shape portion 124 of the braid gradually increasing in diameter in the distal direction, the braid 112 forming a plurality of circumferential rings of cells, a first 126 circumferential ring of cells being disposed at the distal end of the braid, a second 128 circumferential ring of cells being disposed at the distal end of the braid proximal to and adjacent to the first circumferential ring of cells, a third 130 circumferential ring of cells being disposed at the distal end of the braid proximal to and adjacent to the second circumferential ring of cells, a fourth 132 circumferential ring of cells being disposed at the distal end of the braid proximal to and adjacent to the third circumferential ring of cells, a fifth 134 circumferential ring of cells being disposed at the distal end of the braid proximal to and adjacent to the fourth circumferential ring of cells, a sixth 136 circumferential ring of cells being disposed at the distal end of the braid proximal to and adjacent to the fifth circumferential ring of cells, the first, second, third, fourth, fifth, and sixth circumferential ring of cells each being in the tapered shape portion of the braid, the braid comprising a plurality of wires that create the circumferential rings of cells, the distal end 116 of the braid 112 comprising free ends 144;

at least one radiopaque marker 148 disposed about at least one of the free ends 144 of the braid 112; and

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102.

Clause 14. The catheter of Clause 13, wherein the free ends 144 at the distal end of the braid comprising twisted wires 146, the at least one radiopaque marker 148 placed about the twisted wires 146.

Clause 15. The catheter of Clause 14 wherein the at least one radiopaque marker 148 is a sleeve crimped about the distal end of the braid.

Clause 16. The catheter of Clause 14 wherein the at least one radiopaque marker 148/150 is formed by a knot in the braid wire to accentuate the radiopacity of the catheter at the distal end of the braid.

Clause 17. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 made of a wire disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid 112 having a tubular shape portion extending from the proximal end 114 to proximate the distal end 116, the braid 112 having a tapered shape portion 124 at the distal end of the braid, the tapered shape portion 124 of the braid gradually increasing in diameter in the distal direction, the braid 112 forming a plurality of circumferential rings of cells, a first 126 circumferential ring of cells being disposed at the distal end of the braid, a second 128 circumferential ring of cells being disposed at the distal end of the braid proximal to and adjacent to the first circumferential ring of cells, a third 130 circumferential ring of cells being disposed at the distal end of the braid proximal to and adjacent to the second circumferential ring of cells, a fourth 132 circumferential ring of cells being disposed at the distal end of the braid proximal to and adjacent to the third circumferential ring of cells, a fifth 134 circumferential ring of cells being disposed at the distal end of the braid proximal to and adjacent to the fourth circumferential ring of cells, a sixth 136 circumferential ring of cells being disposed at the distal end of the braid proximal to and adjacent to the fifth circumferential ring of cells, the first, second, third, fourth, fifth, and sixth circumferential ring of cells each being in the tapered shape portion of the braid;

at least one radiopaque marker 152 disposed about at least one wire of the braid 112 within one of the first, second, third, fourth, fifth, and sixth circumferential rings of cells; and

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102; wherein the distal end of the braid is spaced proximally from the distal end 106 of the liner 102 and from the distal end of the jacket 118.

Clause 18. The catheter of Clause 17 wherein the at least one radiopaque marker 152 has a planar shape.

Clause 19. The catheter of Clause 18 wherein a thickness tʹ of the at least one radiopaque marker 152ʹ is about half the diameter of the wire forming the braid.

Clause 20. The catheter of any of Clauses 17-19 wherein the at least one radiopaque marker 152ʹ, 152ʺ is folded about the wire of the braid.

Clause 21. The catheter of Clause 20 wherein a thickness tʺ of a middle portion 154 of the at least one radiopaque marker 152ʺ adjacent the wire of the braid is about 25% of the diameter of the wire forming the braid, a thickness tʺʹ of a portion of the at least one radiopaque marker 152ʺ adjacent the middle portion 156 of the at least one radiopaque marker 152ʺ is about 75% of the diameter of the wire forming the braid.

Clause 22. The catheter of Clause 17 wherein the at least one radiopaque marker 158 has a C-shape.

Clause 23. The catheter of Clause 17 wherein the at least one radiopaque marker 160 has a tubular shape.

Clause 24. The catheter of Clause 23 wherein the at least one radiopaque marker 158, 160 is crimped onto the wire.

Clause 25. The catheter of Clause 23 wherein the at least one radiopaque marker 160 has a plurality of circumferential grooves 162 in the center of the tubular shape configured to promote bending of the marker to follow the contour of the wire.

Clause 26. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 made of wire disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid having a tubular shape extending from the proximal end 114 to proximate the distal end, the braid having a tapered shape at the distal end of the braid, the tapered shape of the braid gradually increasing in diameter in the distal direction, the braid forming a plurality of circumferential rings of cells, a first circumferential ring of cells being disposed at the distal end of the braid, a second circumferential ring of cells being disposed at the distal end of the braid proximal to and adjacent to the first circumferential ring of cells, a third circumferential ring of cells being disposed at the distal end of the braid proximal to and adjacent to the second circumferential ring of cells, a fourth circumferential ring of cells being disposed at the distal end of the braid proximal to and adjacent to the third circumferential ring of cells, a fifth circumferential ring of cells being disposed at the distal end of the braid proximal to and adjacent to the fourth circumferential ring of cells, a sixth circumferential ring of cells being disposed at the distal end of the braid proximal to and adjacent to the fifth circumferential ring of cells, the first, second, third, fourth, fifth, and sixth circumferential ring of cells each being in the tapered shape portion of the braid;

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102; wherein the distal end of the braid is spaced proximally from the distal end 106 of the liner 102 and from the distal end of the jacket 118; and

a tip 164, 166 disposed at the distal end of the catheter, the tip connected to the jacket, the tip being made of a polymer material that is impregnated with a radiopaque material.

Clause 27. The catheter of Clause 26 wherein the radiopaque material is one of Tantalum or Tungsten.

Clause 28. The catheter of Clause 27 wherein the tip is made from a thermoplastic polyurethane.

Clause 29. The catheter of Clause 28 wherein the inner layer 166 is impregnated with about 60% to about 90% Tantalum or Tungsten filler.

Clause 30. The catheter of any of Clauses 26-29 wherein the tip is comprised of an inner layer and an outer layer, the inner layer being impregnated with a radiopaque material.

Clause 31. A catheter 100 for navigating within the vasculature of a body, the catheter comprising:

a liner 102 having a proximal end 104, a distal end 106, an outer surface 108, an internal lumen 110 extending from the proximal end 104 to the distal end 106, and a longitudinal axis LA;

a braid 112 disposed on the outer surface 108 of the liner 102, the braid 112 having a proximal end 114 and a distal end 116, the braid extending from the proximal end 104 of the liner 102 to the distal end 106 of the liner 102, the distal end of the braid 116 being spaced from and proximal to the distal end 106 of the liner 102, the braid having a tubular shape extending from the proximal end 114 to proximate the distal end, the braid having a tapered shape at the distal end of the braid, the tapered shape of the braid gradually increasing in diameter in the distal direction, the braid forming a plurality of circumferential rings of cells, a first circumferential ring of cells being disposed at the distal end of the braid, a second circumferential ring of cells being disposed at the distal end of the braid proximal to and adjacent to the first circumferential ring of cells, a third circumferential ring of cells being disposed at the distal end of the braid proximal to and adjacent to the second circumferential ring of cells, a fourth circumferential ring of cells being disposed at the distal end of the braid proximal to and adjacent to the third circumferential ring of cells, a fifth circumferential ring of cells being disposed at the distal end of the braid proximal to and adjacent to the fourth circumferential ring of cells, a sixth circumferential ring of cells being disposed at the distal end of the braid proximal to and adjacent to the fifth circumferential ring of cells, the first, second, third, fourth, fifth, and sixth circumferential ring of cells each being in the tapered shape portion of the braid;

a jacket 118 disposed about the braid 112, the jacket 118 having a proximal end 120 and a distal end 122, the jacket 118 extending from the proximal end 104 of the liner 102 to the distal end 106 of the liner 102, the distal end of the jacket 118 being aligned with distal end 106 of the liner 102; wherein the distal end of the braid is spaced proximally from the distal end 106 of the liner 102 and from the distal end of the jacket 118; and

at least one radiopaque marker 168, 170, 172 disposed between the braid and the jacket within one of the second, third, fourth, fifth, and sixth circumferential rings of cells, the at least one radiopaque marker being disposed in at least two cells of the braid and under a wire of the braid that separates the at least two cells.

Clause 32. The catheter of Clause 31 wherein the at least one radiopaque marker 170 has a symmetrical shape.

Clause 33. The catheter of Clause 31 wherein the at least one radiopaque marker 168, 172 has an asymmetrical shape.

Clause 34. A method of making a catheter 100 for navigating within the vasculature of a body, the catheter comprising:

providing 300 a liner 102 having a proximal end 104, a distal end 106, an outer surface 108, an internal lumen 110 extending from the proximal end 104 to the distal end 106, and a longitudinal axis LA;

etching 302 the outer surface 108 of the liner 102;

providing 304 a braid 112 on the outer surface 108 of the liner 102, the braid 112 having a proximal end 114 and a distal end 116, the braid forming a plurality of diamond shaped cells, the braid extending from the proximal end 104 of the liner 102 to the distal end 106 of the liner 102, the distal end of the braid 116 being spaced from and proximal to the distal end 106 of the liner 102;

placing 306 a first sleeve 174 about the distal end of the braid covering at least a distalmost circumferential row of diamond shaped cells, the first sleeve having a proximal end 176 and a distal end 178, the first sleeve preventing UV light from passing through the sleeve;

placing 308 at least one radiopaque marker 140 on the outer surface of the liner within at least one of the diamond shaped cells adjacent to and spaced a predetermined distance from the distal end of the braid and spaced proximally from the proximal end 176 of the first sleeve 174;

connecting 312 the first sleeve 174 to the distal end 116 of the braid 112;

pulling 314 the first sleeve 174 in the distal direction so the braid 112 is flush with the liner 102;

connecting 310 with an adhesive 180 the at least one radiopaque marker 140 to the liner 102, the at least one radiopaque marker 140 being placed within a circumferential ring of cells proximal of the proximal end 176 of the first sleeve 174;

providing 316 an ultraviolet (UV) light source 182;

curing 318 the adhesive by directing UV light from the UV light source 182 toward the at least one radiopaque marker 140 after the pulling the first sleeve step 314;

placing 320 at least one jacket 118 about the braid 112, the jacket 118 having a proximal end 120 and a distal end 122;

removing 322 the first sleeve 174 from the distal end of the braid 112 after the curing step; and

reflowing 324 the jacket 118 about the braid 112, the at least one radiopaque marker 140, and the liner 102 to form the catheter 100.

Clause 35. The method of making a catheter according to clause 33, further comprising the step of:

placing 326 a second sleeve 184 about the distal end of the braid 112 before the curing step, the second sleeve 184 being spaced proximal of the first sleeve 174 such that the at least one radiopaque marker 140 is disposed between the first sleeve 174 and the second sleeve 184, the second sleeve 184 having a proximal end 186 and a distal end 188, the distal end 188 of the second sleeve being proximal to and spaced from the at least one radiopaque marker 140 on the outer surface of the liner 102, the second sleeve 184 preventing UV light from passing through the sleeve.

Clause 36. The method of making a catheter according to clause 34, further comprising the step of:

removing 328 the second sleeve 184 from the braid 112 after the curing step 318.

Clause 37. The method of making a catheter according to clause 36, wherein the reflowing the jacket about the braid step 324 includes reflowing the jacket 118 from the proximal end 122 of the jacket 118 up to a location proximal of the first sleeve 174.

Clause 38. The method of making a catheter according to clause 37, wherein the step of removing the first sleeve 322 from the distal end of the braid 112 occurs after the reflowing of the jacket 118 about the braid 112 from the proximal end 120 of the jacket 118 to a location proximal of the first sleeve 174.

Clause 39. The method of making a catheter according to clause 38, further comprising the step of:

reflowing 330 of a remaining portion of the jacket about the braid 112 from the location proximal of the first sleeve to the distal end 122 of the jacket 118.

Clause 40. The method of making a catheter according to clause 39, further comprising the step of:

providing 332 a core 190 having a proximal end 192, a distal end 194, and an outer surface 196, wherein the providing a liner step 304 includes placing the liner 102 on the outer surface of the core 190.

Clause 41. The method of making a catheter according to clause 40, further comprising the step of:

trimming 334 the liner 102 and jacket 118 after the reflowing of a remaining portion of the jacket step 330 at a location distal to the distal end of the braid 112.

Clause 42. The method of making a catheter according to clause 41, further comprising the step of:

removing 336 the reflowed liner 102, braid 112, and jacket 118 from the core 190.

Clause 43. The method of making a catheter according to clause 42, further comprising the step of:

placing 338 a tapered mandrel 196 within the distal end of the reflowed liner, braid, and jacket.

Clause 44. The method of making a catheter according to clause 44, further comprising the step of:

placing 340 a tubular sleeve 198 about the tapered mandrel 196, the tubular sleeve 198 having a proximal end 200 and a distal end 202, the proximal end 202 of the tubular sleeve 198 abutting a distal end 204 of the reflowed liner and jacket.

Clause 45. The method of making a catheter according to clause 44, further comprising the step of:

reflowing 342 the tubular sleeve 198 and at least a distal portion of the jacket about the tapered mandrel 196.

Clause 46. The method of making a catheter according to clause 45, further comprising the step of:

trimming 344 the tubular sleeve.

Clause 47. The method of making a catheter according to any one of clauses 35-46, wherein the placing 306 a first sleeve 174 about the distal end of the braid step includes covering at least two distalmost circumferential rings of diamond shaped cells.

Clause 48. The method of making a catheter according to any one of clauses 35-47, further comprising providing the sleeve having a UV protective filler material.

Clause 49. The method of making a catheter according to any one of clauses 35-48, further comprising making the sleeve being from a heat shrink material.

Clause 50. The method of making a catheter according to clause 49, wherein the sleeve is made from a polyethylene terephthalate (PET).

Clause 51. The method of making a catheter according to any one of clauses 35-50, wherein the at least one radiopaque marker 140 has a disc shape.

Clause 52. The catheter of clause 7, wherein the plurality of distal hoops 138 form an atraumatic bend at the distal end of the braid 112.

Clause 53. A method of making a catheter 100 for navigating within the vasculature of a body, the catheter comprising:

providing 300 a liner 102 having a proximal end 104, a distal end 106, an outer surface 108, an internal lumen 110 extending from the proximal end 104 to the distal end 106, and a longitudinal axis LA;

etching 302 the outer surface 108 of the liner 102;

providing 304 a braid 112 on the outer surface 108 of the liner 102, the braid 112 having a proximal end 114 and a distal end 116, the braid forming a plurality of diamond shaped cells, the braid extending from the proximal end 104 of the liner 102 to the distal end 106 of the liner 102, the distal end of the braid 116 being spaced from and proximal to the distal end 106 of the liner 102;

placing 306 a first sleeve 174 about the distal end of the braid covering at least a distalmost circumferential row of diamond shaped cells, the first sleeve having a proximal end 176 and a distal end 178, the first sleeve preventing UV light from passing through the sleeve;

placing 308 at least one radiopaque marker 140 on the outer surface of the liner within at least one of the diamond shaped cells adjacent to and spaced a predetermined distance from the distal end of the braid and spaced proximally from the proximal end 176 of the first sleeve 174;

connecting 312 the first sleeve 174 to the distal end 116 of the braid 112;

pulling 314 the first sleeve 174 in the distal direction so the braid 112 is flush with the liner 102;

connecting 310 with an adhesive 180 the at least one radiopaque marker 140 to the liner 102, the at least one radiopaque marker 140 being placed within a circumferential ring of cells proximal of the proximal end 176 of the first sleeve 174;

placing 320 at least one jacket 118 about the braid 112, the jacket 118 having a proximal end 120 and a distal end 122;

removing 322 the first sleeve 174 from the distal end of the braid 112 after the connecting step 310; and

reflowing 324 the jacket 118 about the braid 112, the at least one radiopaque marker 140, and the liner 102 to form the catheter 100.

54. The method of making a catheter according to clause 47, further comprising the step of: expanding the distal end 204 of the reflowed liner and jacket over the tapered mandrel such that it abuts the sleeve positioned over the enlarged end of the tapered mandrel.

What is claimed is:

1. A catheter for navigating within the vasculature of a body, the catheter comprising:
a liner having a proximal end, a distal end, an outer surface, an internal lumen extending from the proximal end to the distal end, and a longitudinal axis;
a braid formed by at least one wire disposed on the outer surface of the liner, the braid having a proximal end and a distal end, the braid extending from the proximal end of the liner to the distal end of the liner, the distal end of the braid being spaced from and proximal to the distal end of the liner, the braid having a tubular shape extending from the proximal end to proximate the distal end, the braid having a tapered shape portion at the distal end of the braid, the tapered shape portion of the braid gradually increasing in diameter in the distal direction, the braid forming a plurality of circumferential rings of cells, a first circumferential ring of cells being disposed at the distal end of the braid, a second circumferential ring of cells being disposed at the distal end of the braid proximal to and adjacent to the first circumferential ring of cells, a third circumferential ring of cells being disposed at the distal end of the braid proximal to and adjacent to the second circumferential ring of cells, a fourth circumferential ring of cells being disposed at the distal end of the braid proximal to and adjacent to the third circumferential ring of cells, a fifth circumferential ring of cells being disposed at the distal end of the braid proximal to and adjacent to the fourth circumferential ring of cells, a sixth circumferential ring of cells being disposed at the distal end of the braid proximal to and adjacent to the fifth circumferential ring of cells, the first, second, third, fourth, fifth, and sixth circumferential ring of cells each being in the tapered shape portion of the braid;
at least one radiopaque marker disposed within one of the second, third, fourth, fifth, and sixth circumferential rings of cells; and
a jacket disposed about the braid, the jacket having a proximal end and a distal end, the jacket extending from the proximal end of the liner to the distal end of the liner, the distal end of the jacket being aligned with distal end of the liner; wherein the distal end of the braid is spaced proximally from the distal end of the liner and from the distal end of the jacket.

2. The catheter of claim 1, wherein the at least one radiopaque marker disposed within one of the third, fourth, fifth, and sixth circumferential rings of cells.

3. The catheter of claim 1, comprising at least two radiopaque markers positioned within the fourth circumferential ring of cells defined by the distalmost cells.

4. The catheter of claim 3, wherein four radiopaque markers are positioned within the fourth circumferential ring of cells at approximately 90 degrees apart within the fourth circumferential rings of cells.

5. The catheter of claim 1, comprising at least two radiopaque markers positioned within the sixth 136 circumferential ring of cells defined by the distalmost cells.

6. The catheter of claim 5, wherein four radiopaque markers are positioned within the sixth circumferential ring of cells at approximately 90 degrees apart within the sixth circumferential rings of cells.

7. The catheter of claim 1, wherein the distal end of the braid comprising a plurality of distal hoops where the at least one wire of the braid inverts to loop back through the braid and form the circumferential rings of cells.

8. The catheter of claim 1, wherein the at least one radiopaque marker is disc shaped.

9. The catheter of claim 1, wherein the tapered shape portion of the braid is comprised of about thirteen circumferential rings of cells.

10. The catheter of claim 1, wherein the distal end of the braid is spaced proximally from the distal end of the liner and from the distal end of the jacket by approximately 0.50 mm.

11. A catheter for navigating within the vasculature of a body, the catheter comprising:
a liner having a proximal end, a distal end, an outer surface, an internal lumen extending from the proximal end to the distal end, and a longitudinal axis;
a braid disposed on the outer surface of the liner, the braid having a proximal end and a distal end, the braid extending from the proximal end of the liner to the distal end of the liner, the distal end of the braid being spaced from and proximal to the distal end of the liner, the braid having a tubular shape portion extending from the proximal end o proximate the distal end, the braid having a tapered shape portion at the distal end of the braid, the tapered shape portion of the braid gradually increasing in diameter in the distal direction, the braid forming a plurality of circumferential rings of cells, a first circumferential ring of cells being disposed at the distal end of the braid, a second circumferential ring of cells being disposed at the distal end of the braid proximal to and adjacent to the first circumferential ring of cells, a third circumferential ring of cells being disposed at the distal end of the braid proximal to and adjacent to the second circumferential ring of cells, a fourth circumferential ring of cells being disposed at the distal end of the braid proximal to and adjacent to the third circumferential ring of cells, a fifth circumferential ring of cells being disposed at the distal end of the braid proximal to and adjacent to the fourth circumferential ring of cells, a sixth circumferential ring of cells being disposed at the distal end of the braid proximal to and adjacent to the fifth circumferential ring of cells, the first, second, third, fourth, fifth, and sixth circumferential ring of cells each being in the tapered shape portion of the braid, the distal end of the braid comprising distal loops where at least one wire of the braid inverts and forms the circumferential rings of cells;
at least one radiopaque marker disposed within one of the distal loops; and
a jacket disposed about the braid, the jacket having a proximal end and a distal end, the jacket extending from the proximal end of the liner to the distal end of the liner, the distal end of the jacket being aligned with distal end of the liner.

12. The catheter of claim 11, wherein the loops are circular in shape.

13. A catheter for navigating within the vasculature of a body, the catheter comprising:
a liner having a proximal end, a distal end an outer surface, an internal lumen extending from the proximal end to the distal end, and a longitudinal axis;
a braid disposed on the outer surface of the liner, the braid having a proximal end and a distal end, the braid extending from the proximal end of the liner to the distal end of the liner, the distal end of the braid being spaced from and proximal to the distal end of the liner, the braid having a tubular shape extending from the proximal end to proximate the distal end, the braid having a tapered shape portion at the distal end of the braid, the tapered shape portion of the braid gradually increasing in diameter in the distal direction, the braid forming a plurality of circumferential rings of cells, a first circumferential ring of cells being disposed at the distal end of the braid, a second circumferential ring of cells being disposed at the distal end of the braid proximal to and adjacent to the first circumferential ring of cells, a third circumferential ring of cells being disposed at the distal end of the braid proximal to and adjacent to the second circumferential ring of cells, a fourth circumferential ring of cells being disposed at the distal end of the braid proximal to and adjacent to the third circumferential ring of cells, a fifth circumferential ring of cells being disposed at the distal end of the braid proximal to and adjacent to the fourth circumferential ring of cells, a sixth circumferential ring of cells being disposed at the distal end of the braid proximal to and adjacent to the fifth circumferential ring of cells, the first, second, third, fourth, fifth, and sixth circumferential ring of cells each being in the tapered shape portion of the braid, the braid comprising a plurality of wires that create the circumferential rings of cells, the distal end of the braid comprising free ends;
at least one radiopaque marker disposed about at least one of the free ends of the braid; and
a jacket disposed about the braid, the jacket having a proximal end and a distal end, the jacket extending from the proximal end of the liner to the distal end of the liner, the distal end of the jacket being aligned with distal end of the liner.

14. The catheter of claim 13, wherein the free ends at the distal end of the braid comprising twisted wires, the at least one radiopaque marker placed about the twisted wires.

15. The catheter of claim 14 wherein the at least one radiopaque marker is a sleeve crimped about the distal end of the braid.

16. The catheter of claim 14 wherein the at least one radiopaque marker is formed by a knot in the braid wire to accentuate the radiopacity of the catheter at the distal end of the braid.

17. The catheter of claim 7, wherein the plurality of distal hoops form an atraumatic bend at the distal end of the braid.
